(19) United States
(12) Patent Application Publication
(10) Pub. No.: US 2009/0004661 A1
(43) Pub. Date: Jan. 1, 2009

(54) METHOD OF GROWING MESENCHYMAL STEM CELLS FROM BONE MARROW

(75) Inventors: Prathibha Shetty, Maharashtra (IN); Chandra Viswanathan, Maharashtra (IN); Anirban Mallik Thakur, Maharashtra (IN); Geeta Ravindran, Maharashtra (IN)

Correspondence Address: LACKENBACH SIEGEL, LLP, LACKENBACH SIEGEL BUILDING, 1 CHASE ROAD, SCARSDALE, NY, 10583, US

(73) Assignee: Reliance Life Sciences Pvt Ltd., Maharashtra (IN)

(21) Appl. No.: 12/129,898

(22) Filed: May 30, 2008

Related U.S. Application Data
(63) Continuation-in-part of application No. 10/853,077, filed on May 25, 2004.

(30) Foreign Application Priority Data
May 26, 2003 (IN) 532/MUM/2003
Sep. 29, 2007 (IN) 1912/MUM/2007

(51) Int. Cl. C12Q 1/68; C12Q 1/02

(52) U.S. Cl. 435/6; 435/29

(57) ABSTRACT

The present invention provides a method for culturing mesenchymal stem cells using cord blood serum, for therapeutic purposes in regenerative medicine; and in particular the present invention provides for the use of these cells in the treatment of PD, and the present invention has provided proliferation and neuronal differentiation of the MSCs in a xenofree culture system for clinical applications in a simple two step protocol, and the in vivo functional efficacy was tested in Parkinson's animal model.

This Patent Application is based on an Indian patent application and corresponds to an Indian Patent Application serial number 1912/MUM/2007 filed on Sep. 29, 2007 which is a patent of addition to Indian Patent Application serial number 532/MUM/2003 filed on 26 May 2003, and this Application is also a Continuation-in-Part (CIP) of U.S. patent application Ser. No. 10/853,077, filed May 25, 2003.

The present invention provides a method for culturing mesenchymal stem cells (MSCs) using cord blood serum, for therapeutic purposes in regenerative medicine. The present invention in particular provides cultured mesenchymal stem cells for neural regeneration and for use in the therapy of Parkinson's disease (PD), spinal cord injury in animal models and other neurodegenerative diseases.

Bone marrow derived Mesenchymal stem cells (BMMSCs) are a unique population of stem and multipotent progenitor which can be obtained in quantities appropriate for clinical applications, making them good candidates for use in tissue repair. Bone marrow derived mesenchymal stem cells (BMMSCs) is a valid definitive candidate for repair of damaged tissues in degenerative disorders in general and neurological diseases in particular. Neural and embryonic stem cells are extremely versatile but still face several known challenges. In sharp contrast, MSCs have unique plasticity, accessibility and immunosuppressive properties. Translational research needs attention in areas such as up scalability, stability, free from usage of animal products and regulatory compliance, which are soon to be implemented. Techniques for isolation and expansion of mesenchymal stem cells in culture have been established.

Cell replacement therapy aims at grafting therapeutically relevant cells to impaired tissues and has been proposed as future therapies for neurodegenerative disorders. It is well known that neurological diseases like PD, Spinal cord injury, Multiple sclerosis, Alzheimer's disease, Stroke etc. are caused mainly due to the progressive loss of functional cells as a result of either aging, injury besides other several postulated causes. Spontaneous neural tissue repair is known to take place in patients affected by inflammatory and degenerative disorders to a smaller or greater degree. However, this process is not robust enough to promote a functional and long term remission.

### DESCRIPTION OF RELATED PRIOR ART

Recent studies have shown that tissue specific stem cells possess wider transdifferentiation potential than previously thought and can encompass heterologous lineages. There are many reports confirming the neuronal potential of stem cells isolated from adult somatic tissues such as bone marrow [1] (the numbers refer to a list of References referred to under the heading REFERENCES, see paragraph [0031]); hair follicle [2]; amniotic fluid [3]; inner ear [4]; and cornea [5]. BMMSCs (Bone marrow mesenchymal stem cells) are a favorite of stem cell researchers with respect to the transdifferentiation potential especially towards neuronal lineage. [7, 12, 28]. Cytokines, growth factors, neurotrophins, and retinoic acid have been used to promote neural cell induction and differentiation both in vivo and in vitro. Woodbury et al. (see Reference No. 10) have reported usage of chemicals in both rodent and human MSCs for neuronal differentiation in vitro [20, 29]. Publications referred to are listed in paragraph [0031] under References and are also placed in brackets or parenthesis to avoid a detailed repetition of the source. The majority of these exhibited a neuronal morphology and expressed several neuronal markers like NSE (neuron specific enolase), neurofilament-M, tau, and NeuN [10].

Amongst the various neurological disorders, PD is a chronic, progressive neurodegenerative movement disorder. Tremors, rigidity, slow movement (bradykinesia), poor balance, and difficulty in walking (called parkinsonian gait) are major characteristic symptoms of PD. PD results from the degeneration of dopamine-producing nerve cells in the brain, specifically in the substantia nigra and the locus coeruleus. The disease burden is reported to be huge. Approximately 5-6 million people are affected globally. The prevalence varies widely from 82 per 100,000 in Japan and 108 per 100,000 in UK, to nearly 1% (approximately 1 million) of the population in North America. In India, however the prevalence rate of PD is highest in the Parsi community in Western India. (363 per 100,000) followed by other parts of the country which is 14 per 100,000 in North India, 27 per 100,000 in South India, 16 per 100,000 in East India.

PD is a neurodegenerative disorder characterized by loss of midbrain dopaminergic neurons in the substantia nigra. It is well known that L-dihydroxyphenylalanine (L-DOPA) can attenuate motor dysfunctions, but long-term efficacy of this treatment gradually decreases over time with multiple side effects. Cell replacement therapy to restore the degenerated dopaminergic neurons may serve as a viable alternative to achieve significant clinical improvement. Cell-based therapies derived from fetal or embryonic origin have been tested with questionable success. Yet, technical, ethical, practical, limited availability, variable outcomes, continue to be the researcher's nightmare [6].

Under these circumstances, adult stem cells could be an ideal source for cell replacement therapy due to their self-renewal and multilineage developmental potentials. Because of their unique attributes of plasticity and accessibility, BMMSCs are a definite alternative to neural or embryonic cells in replacing autologous damaged tissues for several neurodegenerative diseases. By harnessing the neuronal potential of readily available and accessible adult bone marrow cells, substantial ethical and technical dilemmas may be circumvented. Recent studies have shown that BMMSCs improve neurological deficits when transplanted into animal models of neurological disorders. The transdifferentiation potential of MSCs into neurons in vitro has been reported earlier [9, 10, 27, 28].

PD is a chronic and a progressive neurodegenerative movement disorder. Tremors, rigidity, slow movement (bradykinesia), poor balance, and difficulty walking (called parkinsonian gait) are characteristic primary symptoms of PD. PD results from the degeneration of dopamine-producing nerve cells in the brain, specifically in the substantia nigra and the locus coeruleus Dopamine is a neurotransmitter that stimulates motor neurons, those nerve cells that control the muscles. When dopamine production is depleted, the motor system nerves are unable to control movement and coordination.

While the initial treatment with L-dihydroxyphenylalanine (L-DOPA) can attenuate motor dysfunctions, the long-term efficacy of the treatment gradually decreases over time with multiple side effects. Cell replacement therapy to replace the degenerated dopaminergic neurons may serve as an alternative to achieve significant clinical improvement. Traditionally, cell-based therapies for the CNS have been derived from fetal or embryonic origin. Fetal cell transplantation has significant technical, ethical and practical problems partly due to limited availability and variable outcomes [G. Freed et al 2001, 6].

Stem cells could be an ideal source for cell replacement therapy due to their self renewal capacity and multilineage developmental potential. Because of their unique attributes of plasticity and accessibility, bone marrow-derived mesenchymal stem cells (MSCs) may serve as a valid alternative to neural or embryonic cells in replacing autologous damaged tissues for neurodegenerative diseases. By harnessing the neural potential of readily-available and accessible adult bone marrow, substantial ethical and technical dilemmas may be circumvented. BMMSCs offer the best hope for autologous stem cell based replacement therapies because of their potency, accessibility and immunosuppressive properties.

They are a unique population of multipotent progenitor cells which can be obtained in quantities adequate for clinical applications, thus making them good candidates for use in tissue repair including our own reports about isolation and expansion of MSCs in cultures; however, these were done in FBS our own earlier publications. Feasibility and safety of the application of BMMSC for clinical use propagated ex vivo in FBS containing cell cultures, has been documented in a significant number of studies over the last decade. [Ringden et al, 2006, 16]. In this respect, MSCS free of animal components to be safe for regenerative medicine, Briefing again: We have injected BMMSC cultured in CBS for neuronal progenitors and injected in to animals to check whether these actually transdifferentiated into neuronal cells. The data which is not shown is the behavioral effects which cannot be measured but only recorded as a video clip. To ensure that these BMMSC have the capacity to transdifferentiate into neuronal precursors, we have done in vitro (lab experiments) to give the proof of concept for the neural precursors.

However, the use of FBS (Fetal Blood Serum) during MSC propagation carries the risk of transmission of known and unknown pathogens as well as xenoimmunization, which is an important issue to be addressed [18, 19]. Attempts have been made by several groups for replacing FBS with growth factors derived by mixing purified factors which are either isolated from FBS or a mixture of growth factors derived by recombinant methods. However, these culture media have their associated shortcomings and risks since they are unable to support MSCs (Mesenchymal stem cells) expansion beyond 2 passages. [Meuleman et al, 2006, 14].

Use of autologous serum in the culture media is a better option for addressing this issue. Mizuno et al (2006) [15] used autologous human serum for expanding BMMSCs for 9 days, which gives limited expansion, not adequate enough for clinical use. This can still be considered a good option in certain limited clinical conditions but in a larger perspective of clinical conditions, obtaining autologous serum in adequate quantities will be a major challenge to the manufacturers. Further the various limitations of the donors such as aging, disease conditions, logistics etc poses challenges on using autologous serum as a better option.

In the previous parent patent application of which this Application is a CIP, filed May 25, 2003 under Ser. No. 10/853,077, the inventors in that application, one of which is in common in this Application, had demonstrated that culturing of MSC (Mesenchymal stem cell) isolated from human Bone Marrow aspirate in the presence of human umbilical cord blood serum instead of FBS promotes more effective expansion and also retains their differentiation capacity. The above-noted parent application which is incorporated herein by reference has previously shown the superiority of using cord blood serum as a xenofree alternative to FBS. Under these circumstances, the inventors could expand MSC with no undesirable effect whatsoever on transdifferentiation and stability in cultures for more than 5 passages. The Applicants therefore could generate large quantities of BMMSCs that meets the clinical requirements.

In the Parent application Ser. No. 10/853,077, the subject matter of which is incorporated by reference as if the entire text were included herein, prior art was brought to the attention of the applicants in that application and the assignee, and such prior art while of interest in the view of the Applicants and assignee of this Application, such prior art did not anticipate the subject matter as disclosed in the Parent Application nor render the subject matter disclosed as obvious.

Sanberg et al., U.S. Pat. No. 7,160,724 appears to teach that bone marrow is a common source of stem cells, and has given examples related to MNC from UCB. Sanberg et al. does not teach growing cord blood stem cells isolated from MNC of cord blood and proliferated in a medium of DMEM/Ham's F12, glutamine and sodium bicarbonate and Sanberg et al. was considered to teach that a bone marrow is a common source of stem cells. Sanberg et al. U.S. Pat. No. 7,160,724 is of interest because it teaches that bone marrow is a common source of stem cells, and has given examples related to MNC from UCB. There is no disclosure in Sanberg et al. about growing cord blood stem cells isolated from MNC of cord blood and proliferated in a medium of DMEM/Ham's F12, glutamine and sodium bicarbonate and further Sanberg et al. does not disclose that bone marrow is a common source of stem cells. The mere fact that it is possible to consider the possibility of therapeutic use does not render this disclosure as obvious. Usually bone marrow or cord blood is a rich source of stem cells. Umbilical cord blood is depleted of RBC and the leukocyte rich fraction is subjected to density gradient separation Aspirates from bone marrow is subjected to density gradient to yield MNC. Typically, MNCs comprise hematopoietic and non-hematopoietic cells. Sanberg et al. does not disclose nor teach that one can replace fetal bovine serum with cord blood serum in order to have better proliferation kinetics. Sanberg et al. has taught that the mononuclear cell fraction is grown in the presence of fetal bovine serum whereas the present invention teaches and discloses that cells are grown in CBS. Sanberg et al. does not disclose nor teach that the cell can express CD73 and CD105 markers. The Sanberg et al. patent does not mention that these are mesenchymal stem cells. All that Sanberg et al. does is to mention the source of neural precursors from umbilical cord blood. The depleted RBC and the MNC fraction is then subjected to a selection of non-hematopoietic cells and these non-hematopoietic cells are then differentiated in medium that allows the cells to become neural cells. Clearly, there is no teaching that the non-hematopoietic cells are mesenchymal stem cells nor are they characterized by the markers of the present invention. According to the abstract of Sanberg et al., use is made of umbilical cord blood for the repair of neural tissue and to treat neurodegenerative diseases of the brain and spinal cord among others, primarily for neural tissue. The Sanberg et al. disclosure is broad enough to provide a basis for a universal case for all diseases without specificity. Since markers are known in the art, and Sanberg et al. does not suggest or disclose the use of markers, it is of little relevance to the work done and contributed in this Application. Sanberg et al. does not teach that non-hematopoietic cells are mesenchymal stem cells. Sanberg et al. teaches the culturing of non-hematopoietic stem cell. Sanberg et al. does not define the non-hematopoietic stem cells except that these are from cord blood. It is not clear if these are MSC, Unrestricted Somatic Stem Cells (USSC) or other types of stem cells. It should also be noted that the Sanberg et al. patent is related to just CD34-cells which means that CD34 negative implies non-hematopoietic cells. Sanberg et al. does not teach that one can replace fetal bovine serum with cord blood serum in order to have better proliferation kinetics. Sanberg et al. teaches that the mononuclear cell fraction is grown in presence of fetal bovine serum and the present Application as will be explained demonstrates that cells are grown in CBS. There is no teaching in Sanberg et al. that the cell can express CD73 and CD105 markers. The Sanberg et al. patent does not mention nor teach that these are mesenchymal stem cells. The characterization of MSCs is not provided in the Sanberg et al. patent, and it is not a source of neural precursors from umbilical cord blood. The RBC fraction is depleted, and the MNC fraction is then subjected to selection of non-hematopoietic cells and these non-hematopoietic cells are then differentiated in a medium that allows the cells to become neural cells. Therefore, Sanberg et al. does not teach that the non-hematopoietic cells are mesenchymal stem cells nor are they characterized by the markers of the present invention.

In the Parent Patent Application, the reference Buhring et al. [48] was cited by the Examiner for CD markers. In this respect, it would appear that Buhring et al. teaches other names for markers CD73 and CD105. Moreover, other than the use of other names for markers, there does not appear to be anything related to Parkinson's disease. Buhring et al. [48] is concerned with osteogenesis imperfecta, cartilage repair, and myocardial infarction. Further, Buhring et al. [48] just directs these markers, but does not provide a full analysis of these markers. According to Buhring et al., [48] prior to their paper, CD271 was described as the most selective marker for the characterization and purification of human BM-MSC. In particular, 271+ cells were used in the example provided, and detection was limited to 271^{bright} cells. It should also be noted that while Buhring et al. [48] teaches names for these markers, it does not teach this invention. Buhring et al. [48] indicates the difficulty involved with respect to the use and selection of markers. Buhring et al. [48] clearly sets forth that at the time of writing of their paper, only a few markers which have been developed and proven to be suitable for the isolation of mesenchymal stem cells from primary tissue. Certain specific markers are indicated which meet the specific established criteria for their positive selection. Also, to identify new MSC-specific markers more than two hundred (200) antibodies were screened. Markers are conventionally further identified as plus and/or minus and have specific unique purposes and the generic designation does not mean that the specific sub-generic Marker will also be useful for all purposes. Buhring et al. [48] clearly indicates the necessity for further work to identify suitable Markers.

Hariri, U.S. Pat. No. 7,311,905 [45] is concerned with embryonic-like stem cells that originate from a post-partum placenta with conventional cord blood compositions or other progenitor cells. This disclosure is primarily concerned with stem cells that can or may be mixed with other stem cells populations. These stem cells are disclosed as being capable of treating vascular and neurological diseases among others. While there is no specific reference to the use of mesenchymal stem cells, prior art dealing with mesenchymal stem cells is discussed. This disclosure is primarily concerned with obtaining stem cells which are drained of cord blood and flushed to remove residual blood. This patent to Hariri [45] is further concerned with combining embryonic stem cells with cord blood. The fact that Hariri discloses certain cells which may be pertinent to the subject matter of this Application still does not teach the invention. The Hariri's [45] Embryonic Like Stem Cells also express Oct-4 which is a well known embryonic stem cell marker. In fact, Hariri [45] calls his cells embryonic because they express Oct-4. Therefore, Hariri's [45] cells are CD73±/1-5±/45−/Oct-4+ whereas as will be pointed out in the detailed description, the cells in the present invention are CD73+/105+/45−/Oct-4−. Therefore, the MSC which are described in this Application are different from Hariri's [45] Embryonic Like Stem Cells. The condition for growing one type of stem cells cannot be obviously applied to another type of stem cells because Hariri's [45] U.S. patent disclosure does not teach that cord blood serum can be used for growing MSC. The teachings of Hariri [45] are not related to embryonic stem cells and do not disclose and do not specifically discuss mesenchymal stem cells. The teachings of this Application are related mesenchymal stem cells and the teachings of Hariri [45] cannot be extrapolated to mesenchymal stem cells. Conditions for growing hematopoietic stem cells cannot be used for growing mesenchymal stem cells although both are stem cells and are found in the marrow. Hariri's [45] teachings therefore cannot be extended to mesenchymal stem cells without further explanation. Hariri [45] does not explain nor teach that cord blood serum has the capacity to expand and that the proliferation kinetics is better in CBS instead of FBS. Clearly, a simple change in the source of serum can have an effect on the kinetics of the cells, which cannot be predicted with exactness. Clearly, it is not possible to simply modify the conditions of embryonic like cells and non-hematopoietic cells and have such teachings applied to mesenchymal stem cells.

Falkenburg, EP Patent 1099754 [43], is of interest but there is no disclosure that cord blood stem cells can read on mesenchymal stem cells. Falkenburg [43] indicates that MSC are CD34+ and CD45−. As CD34+ is a specific marker for hematopoietic stem cells, the mesenchymal stem cells of Falkenburg [43] are from hematopoietic stem cells. The MNC fraction from UCB and BM comprises hematopoietic and non-hematopoietic stem cells which are determined by CD34 marker. With respect to the pluripotency of cells for defining the mesenchymal stem cells. The phenotype of the cells and its source is important for characterization of the MSC. According to Falkenburg [43] all cells from adult bone marrow express CD34 and CD45 and only a fraction of CD34+ cells in UCB and fetal bone marrow express CD45 and this differs from the present invention which is related to MSC from the non-hematopoietic fraction, i.e. from CD34− cells. Falkenburg EP 1099754 [43] is concerned with MSC and CD34+ and CD45−. CD34+ is disclosed as a specific marker for hematopoietic stem cells. The mesenchymal stem cells of Falkenburg [43] are from hematopoietic stem cells. The MNC fraction from UCB and BM comprises both hematopoietic and non-hematopoietic stem cells which are determined by CD34 marker. As will be pointed out in the detailed discussion of the invention, the present Application deals with and is concerned with the non-hematopoietic fraction.

Pittenger et al., Patent Publication No. 2003/0103951 [42], while concerned with MSC's in connection with the heart of a mammal and heart valves, clearly which does not teach isolation methods of MSC from swine bone marrow. There is a general disclosure that the allogenic donor MSC were isolated from swine bone marrow and expanded in culture. This is not a teaching or direction that MSC can be isolated from bone marrow with the characterization as explained in this Application. This is to be contrasted with one of the main aims of the present invention which is to expand the CD45− and CD73 and 105 positive cells (MSC) from bone marrow and cord blood in a media containing cord blood serum. The MSC of the present invention are not embryonic like by providing the Oct-4 expression.

Nandoe et al. [50] refers to Bone Marrow Stromal cells and their use in repair of spinal Cord, in this article the Bone marrow stromal cells which includes all MSC and other cells. They themselves have written in the page 564, first column, 3^{rd} paragraph that there is confusion about the terminology. These cells are a heterogenous population of cells and the isolation techniques do not involve the density gradient separation. Hence, there are chances of variation in the activity of the cells every time. The information provided in this publication is proof that with the help of density gradient separation and analysis of the cells using the markers, the results are of more reliable and reproducible.

The Tomita et al. [49] paper refers to the inducing the Bone marrow mesenchymal stem cells into cardiomyocytes with 5-azacytidine before the injection into the animals/recipient. However, the present invention does not require the induction into differentiated cells. The neural precursors were induced ex vivo to provide the proof of concept.

Usually bone marrow or cord blood is considered to be a rich source of stem cells. Umbilical cord blood is depleted of RBC and the leukocyte rich fraction is subjected to density gradient separation. Aspirates from bone marrow are subjected to density gradient to yield MNC. Typically, MNCs comprise hematopoietic and non-hematopoietic cells.

The present invention deals with CD34− ve cells and that are CD45−. Hence, these cells are not the same as CD34+ of Sanberg et al. [44] or of Falkenburg [43]. The present Application deals with non embryonic like stem cells in that they do not express Oct-4.

### DISTINCTIONS FROM PRIOR ART

The present invention has found and teaches that cord blood serum has the capacity to expand and that the proliferation kinetics is better in CBS instead of FBS. This itself explains that a simple change in the source of serum can have an effect on the kinetics of the cells, and that proves that it is not an ordinary work of a person skilled in the art to just modify the conditions of embryonic like cells and non-hematopoietic cells to be applied to mesenchymal stem cells.

The present application provides the use of CBS (Cord Blood Serum) for expansion by approved validated protocols. CBS is processed as per available regulatory guidelines in controlled cGMP environment, using excipients that can satisfy quality parameters. The present invention also intends to provide that bone marrow derived MSC cultured under xeno free conditions continued to maintained the mesenchymal surface marker expression and displayed a typical mesenchymal phenotype CD73+/CD105+/CD44+/CD29+/SSEA4+/CD45−/CD31−/vWF−/CD14−.

The present invention is an improvement of the invention disclosed and claimed in U.S. Pat. No. 7,060,494 and owned by the assignee of this Application and is designated by the assignee as Reliance P-107. Briefly, this Patent is also designated as Relia P-107.

The present invention deals with CD34− ve cells and that are CD45−. Hence, these cells are not the same as CD34+ of Sanberg et al. [44] or of Falkenburg et al. [43]. The present Application deals with non embryonic like stem cells in that they do not express Oct-4.

The same type of stem cells from different animals can also have different growth requirements. For e.g. mouse embryonal stem (ES) cells require Leukemia inhibitory factor (LIF) for maintaining their undifferentiated state in the absence of feeders. However human ES cells cannot maintain their undifferentiated state in the presence of LIF alone in feeder free cultures (Odorico, Stem Cells, 2001; 19:193-204). Therefore, culture conditions developed for swine MSC may not necessarily be applicable for human MSC. Supporting data in this patent application clearly shows that cord blood serum can be used for growing mesenchymal stem cells from both human and swine marrow. This is one of the novel features of this invention.

### REFERENCES

1. Long X, Olzewksi M, Huang W, et al: Neural cell differentiation in vitro from adult human bone marrow mesenchymal stem cells. Stem Cells Dev14, 65-9 (2005).
2. Seiber-Blum M, Grim M, Hu Y. F, et al.: Pluripotent neural crest stem cells in the adult hair follicle. Dev. Dyn 231, 258-269 (2004).
3 Prusa A R, Marton E, Rosner M, et al.: Neurogenic cells in human amniotic fluid. Am J Obstet Gynecol 191(1), 309-14 (2004).
4. Li H, Liu H., Heller S: Pluripotent stem cells from the adult mouse inner ear. Nat. Med. 9, 1293-1299 (2003).
5. Seigel, G. M., Sun W, Salvi R, et al.: Human corneal stem cells display functional neuronal properties. Mol. Vision. 30, 159-163 (2003).
6. Freed, C. R. Greene P. E., Breeze R. E., et al.: Transplantation of embryonic dopamine neurons for severe PD. N. Engl. J. Med 344, 710-719 (2001).
7. Kan I, Ben-Zur T, Barhum Y et al.: Dopaminergic differentiation of human mesenchymal stem cells—utilization of bioassay for tyrosine hydroxylase expression. Neurosci Lett May 23 419(1), 28-33 (2007). Epub Apr. 13 (2007).
8. Hellmann M A, Panet H, Barhum Y et al.: Increased survival and migration of engrafted mesenchymal bone marrow stem cells in 6-hydroxydopamine-lesioned rodents. Neurosci Lett March 6; 395(2), 124-8 (2006). Epub Dec. 15 (2005).
9. Cho K J, Trzaska K A, Greco S J et al.: Neurons derived from human mesenchymal stem cells show synaptic transmission and can be induced to produce the neurotransmitter substance P by interleukin-1 alpha. Stem Cells March 23(3), 383-91 (2005).
10. Woodbury D, Schwarz E J, Prockop D J et al.: Adult rat and human bone marrow stromal cells differentiate into neurons. J Neurosci Res August 15; 61(4), 364-70 (2000).
11. Wang T T, Tio M, Lee W et al.: Neural differentiation of mesenchymal-like stem cells from cord blood is mediated by PKA. Biochem Biophys Res Commun June 15; 357(4), 1021-7 (2007). Epub Apr. 18 (2007).
12. Choi C B, Cho Y K, Prakash K V et al.: Analysis of neuron-like differentiation of human bone marrow mesenchymal stem cells. Biochem Biophys Res Commun November 10; 350(1), 138-46 (2006). Epub Sep. 14 (2006).
13. Deng J, Petersen B E, Steindler D A et al.: Mesenchymal stem cells spontaneously express neural proteins in culture and are neurogenic after transplantation. Stem Cells April 24(4), 1054-64 (2006). Epub Dec. 1, 2005).
14. Meuleman N, Tondreau T, Delforge A et al.: Human marrow stem cell culture: serum free medium allows better xpansion than classical a-MEM medium. Eur J Haematol 76, 309-16 (2006).
15. Mizuno N, Shiba H, Ozeki Y et al.: Human autologous serum obtained using a completely closed bag system as a substitute for fetal calf serum in human mesenchymal stem cell cultures. Cell Biol Int30, 521-4 (2006).
16. Le Blanc K, Rinden O: Mesenchymal stem cells.: properties and role in clinical bone marrow transplantation. Curr. Opin. Immunol Octl 8(5), 586-91 (2006). Epub Aug. 1, 2006).
17. Tondreau T, Lagneaux L, Dejeneffe M et al: Bone marrow-derived mesenchymal stem cells already express specific neural proteins before any differentiation. Differentiation 72 (7), 319-326 (2004).
18. Edwin M. H, Patricia L. G, Winston K. K et al.: Isolated allogeneic bone marrow-derived mesenchymal cells engraft and stimulate growth in children with osteogenesis imperfecta: Implications for cell therapy of bone. PNAS June 25, 99 (13), 8932-8937 (2002).
19. Carl A. G, Emigdio R, Mandolin J. W et al.: Enhanced Engraftment of Mesenchymal Stem Cells in a Cutaneous Wound Model by Culture in Allogenic Species-Specific Serum and Administration in Fibrin Constructs. Stem Cells 24 (10), 2232-2243 (2006).
20. Karen B, Susanne K, Herald K et al.: Critical Parameters for the isolation mesenchymal stem cells from umbilical cord blood. Stem Cells 22, 625-634 (2004).
21. Mark F. P, Alastair M. M, Stephen C. B et al.: Multilineage Potential of Adult Human Mesenchymal Stem Cells. Science 284 (5411), 143-147 (1999).
22. Hellmann M. A, Panet H, Barhum Y et al.: Increased survival and migration of engrafted mesenchymal bone marrow stem cells in 6-hydroxydopamine-lesioned rodents. Neuroscience letters 395 (2), 124-128 (2006).
23. Nagwa S. El-Badri, Amal H et al.: Cord Blood Mesenchymal Stem Cells: Potential Use in Neurological Disorders. Stem Cells and Development 15(4), 497-506 (2006).
24. Tanavde V, Malehom M, Lumkul R et al.: Human stem progenitor cells from neonatal cord blood have greater haematopoeitic expansion capacity than those from mobilized adult blood. Exp Hematol 30, 816-23 (2002).
25. Shetty P, Bharucha K and Tanavde V.: Human umbilical cord blood serum can replace fetal bovine serum in the culture of mesenchymal stem cells. Cell Biology International 31, 293-298. (2007).
26. Ravindran G, Rao H.: Enriched NCAM-Positive cells form functional dopaminergic neurons in rat model of PD. Stem Cells and Development 15(4), 572-582 (2006).
27. Sanchez-Ramos J R.: Neural cells derived from adult bone marrow and umbilical cord blood. J Neurosci Res September 15; 69(6), 880-93 (2002).
28. Krampera M, Marconi S, Pasini A et al.: Induction of neural-likedifferentiation in human mesenchymal stem cells derived from bone marrow, fat, spleen and thymus. Bone 40(2), 382-90 (2007).
29. Chen Y, Teng F. Y. H, Tang B. L et al.: Coaxing bone marrow stromal stem cells towards neuronal differentiation: progress and uncertainties. Cellular and Molecular Life Sciences 63(14), 1649-1657 (2006).
30. NOORT W A ET AL: “Comparison of repopulating ability of hematopoietic progenitor cells isolated from human umbilical cord blood or bond marrow cells in NOD/SCID mice.” BONE MARROW TRANSPLANTATION, (1998 July) 22 SUPPL 1 S5860, XP000929915, abstract, page S59; FIG. 1A
31. LUI X ET AL.: “Human mesenchymal stem cells enhance ex vivo expansion of human megakaryocyte, erythroid and myeloid progenitors from purified cord blood CD34+ cells”
32. BLOOD: Vol. 92, no. 10 part 1, 15 Nov. 1998 (1998 Nov. 15), page 2977 XP002114346, abstract
33. BAUM C M ET AL.: “Isolation of a candidate human hematopoietic stem-cell population.” PROCEEDINGS OF THE NATIONAL ACADEMY OF SCIENCES OF THE UNITED STATES OF AMERICA, (1992 Apr. 10 89 (7) 2804-8, XP002144544, abstract
34. WO 95 25164 A: (THE PICOWER INSTITUTE FOR MEDICAL RESEARCH) 21 Sep. 1995 (1995 Sep. 21), page 26; table 1, claims 1-33
35. LEBIEN T W ET AL: “Multiparameter flow cytometric analysis of human fetal bone marrow B cells.” LEUKEMIA, (1990 MAY) 4 (4) 354-8, XP000929907, page 357, right-hand column, line 11-line 14
36. WALLER E K ET AL: “The common stem cells” hypothesis reevaluated: human fetal bone marrow contains separate populations of hematopoietic and stromal progenitors.” BLOOD, (1995 May 1) 85 (9) 2422-35, XP000929998, abstract
37. WO 99 64566 A: (OSIRIS THERAPEUTICS) 16 Dec. 1999 (19999 Dec. 16), page 5, line 24-line 26; claims 1-11
38. WO 95255164 A: Publication date 21 Sep. 1995
39. WO 9964566 A: Publication date 16Dec. 1999
40. BHAT, Aravind Venkatrao: U.S. 2003/0232432, Patent Publication
41. PITTENGER, et al.: U.S. 2003/0103951, Patent Publication
42. KIM, et al.: U.S. 2004/0151701, Patent Publication
43. FALKENBURG, Johan: EP 1 099 754 A1
44. SANBERG et al.: U.S. 2002/0028510, U.S. Pat. No. 7,160,724
45. HARIR1, Robert J.: US 2003/0180269, U.S. Pat. No. 7,311,905
46. RAI, Prathibha: U.S. 2004/0203142
47. Human umbilical cord blood serum replace fetal bovine serum, P. Shetty, et al. article by Cell Biology International, xx (2006) 1-6, in India
48. Buhring et al.: Novel Markers for the prospective isolation of human MSC. Annals of NY Academy of Sciences, 2007
49. Tomila, Yurcho et al.; Application of mesenchymal stem cell-device cardiomyocytes as bio-pacemakers; current status and problems to be solved; Med Bio Eng. Coput (2007) 45:209-2; DO/10 1007/s 11517-007-0163-4 30 Jan. 2007
50. Nandoe, Rishi D. S. et al.; Bone Marrow Stromal Cells for Repair of the Spinal Cord; Towards Clinical Application; Cell Transplantation, Vol. 15, pp. 563-577, 2006

The above list of related art and information is referred to by reference to the listing number as well as by further identification in certain parts of the specification.

SYMBOLS

| | |
|---|---|
| (MSCs) | Mesenchymal stem cells |
| (BMMSCs) | Bone Marrow mesenchymal stem cells |
| NSE | Neuron specific enolase |
| PD | Parkinson's disease |
| L-DOPA | L-dihydroxyphenylalanine |
| CNS | Central nervous system |
| FBS | Fetal Blood Serum |
| MNC | Mononuclear Cell |
| UCB | Umbilical cord blood |
| RBC | Red blood cells |
| CBS | Cord Blood Serum |
| cGMP | Current good manufacturing practices |
| BM | Bond marrow |
| DMSO/BHA | Chemical inducers - DMSO: Dimethyl sulfoxide |
| | BHA: Butylated Hydroxyanisole |
| NGF | Nerve growth factors |
| TH | Tyrosine hydroxylase: An enzyme responsible for catalyzing |
| | the conversion of the amino acid L-tyrosine to |
| | dihydroxyphenylalanine (DOPA) |
| β tubulin | Microtubule element expressed exclusively in neurons, and is |
| | a popular identifier specific for neurons in nervous tissue |
| GLP | Glucagon-like peptide-1 Page 17; #5 |
| DMEM | Dulbecco's modified Eagle's medium |
| hMSC | Human mesenchymal stem cell |
| β-FGF | Fibroblast Growth Factor (FGF) |
| CD | Cluster of differentiation: used for the identification and |
| | investigation of cell surface molecules present in leukocytes |
| MHC | Major histocompatibility complex |
| DMSO | Dimethyl sulfoxide |
| BHA | Butylated Hydroxyanisole |
| RP-HPLC | Reverse Phase High Pressure Liquid Chromatography |
| 7AAD | 7-Aminoactinomycin D |
| NF-70 | Neurofilment-70 |
| NeuN | Neuron specific nuclear protein |
| TH | Tyrosine Hydorxylase |
| GFAP | Glial Fibrillary Acidic Protein |
| RT-PCR | Reverse Transcriptase PCR |
| UD | Undifferentiated cell |
| D | Differentiated cell |
| NTERA 2D | NTera-2 is a human cell line which is neuronally-committed |
| | cell |
| Alexa 568 | Human nuclei |
| FITC | Fluorescein isothiocyanate: a derivative of fluorescein used |
| | in wide-ranging application including flow cytometry |
| EGF | Epidermal growth factor |
| FGF | Fibroblast Growth Factor |
| TGF-β | Transforming growth factor-β |
| IMDM | Iscoves modified Dulbeccos medium |
| RPMI | Roswell Park Memorial Institute medium |
| MSCGM | Mesenchymal Stem Cell Growth Medium |
| 6-OHDA | 6-hydroxydopamine |
| vWF | Endothelial markers |
| CD34, CD45 | Hematopoietic marker |
| CD73, Cd105, Cd44, CD29 | Mesenchymal stem cells |
| SSEA4 | Undifferentiated marker |
| HLA ABC | MHC class I |
| HLA DR | MHC class II |
| CD73 | Stem cell marker, endothelial and epithelial |
| CD105 | Undifferentiated marker |
| CD14 | Marker |
| CD29 | Marker |
| CD31 | Marker |
| ECD | Electron-capture detector |
| RNA | Ribonucleic acid |
| NFH | neurofilament proteins heavy |
| NFM | neurofilament proteins intermediate |
| DiI | Molecular proteins |
| NANOG | Marker |
| NURRL | An orphan receptor of the nuclear receptor superfamily, is |
| | widely expressed in the central nervous system (CNS) |
| | including brain regions where dopaminergic neurons are |
| | abundant |
| BRL | Gibco |
| EDTA | Trypsin |
| FICOLL ™ | Ficoll is a registered trademark owned by GE Healthcare |
| | companies |
| | Used in biology laboratories to separate blood to its |
| | components (erythrocytes, leukocytes, etc.) |
| PERECOLL ™ | Percoll is a registered trademark of GE Healthcare |
| | Used for the isolation of cells, organelles, viruses by density |
| | centrifugation |
| EGP | Epidermal growth factor |

### OBJECTS OF THE INVENTION

It is an object of the present invention to provide process conditions for proliferation of BMMSCs using xenofree media

It is another object of the present invention to check the differentiation capacity of the BMMSCs cultured in CBS in a regulated environment and assess their in vitro and in vivo neurogenic potential.

It is a further object of the present invention to study the genotypic, phenotypic and functional properties of CBS cultured and expanded BMMSCs.

It is still another object of the present invention to assess the therapeutic potential of the CBS cultured BMMSC in Parkinson's' diseases.

The present invention has shown that BMMSCs expanded in the presence of medium containing CBS maintain their neural differentiation potential in vitro and also have the ability to show functional improvement after transplantation in PD rat brains, thus making it useful for infusion into humans making it important in the field of regenerative medicine.

In one aspect, the present invention has provided methods for expanding the bone marrow derived MSCs under xeno free conditions. We analyzed the proliferative potential of BMMSCs and confirmed the phenotype by flow cytometry.

In another aspect, the present invention has provided methods for characterization of the bone marrow derived cells cultured in CBS. In one preferred aspect the present invention has provided the genotypic, phenotypic and functional characterization of the cells.

In yet another aspect, the present invention has provided methods of assessing the differentiation capacity of the xenofree cultured BMMSCs. In a preferred aspect, the differentiation was induced by neuronal induction media supplemented with Butylated hydroxy anisole, protein kinase activator and growth factors such as NGF (Nerve growth factors) and Noggin. Differentiated cells were characterized at cellular and molecular levels.

In one aspect, the present invention provides methods for demonstrating the in vitro and in vivo neuronal potential.

In summary, the present invention demonstrates that BMMSCs are a good potential source for treatment of PD. Since these cells have been grown in CBS they overcome the hurdle of infusion into a human for its clinical applications. The present invention has also proved to be an improvement over the disclosure in the parent application Ser. No. 10/853,077 by providing a better and feasible source of serum, which not only allows expansion of MSCs but also maintains and retains into differentiation potential.

Thus, the present invention relates to the methods for providing MSCs free of animal components thus proving to be safe for regenerative medicine and cell therapy applications which will help in treatment of a variety of neurological disorders with great deal of clinical applications. Functionality was evaluated estimating dopamine secretion. 6-OHDA lesioned PD models that stereotaxically received clinical grade MSCs were assessed for 3 months.

The present invention thus has demonstrated the dopaminergic differentiation capabilities by BMMSCs cultured in CBS. This was confirmed by the expression of TH (Tyrosine hydroxylase) at the cellular and molecular level. The measurement of dopamine secreted in the culture supernatant confirms the functionality. In addition to TH, the differentiated cells have shown positivity for other neuronal markers as well, such as NeuN (Neuron specific nuclear protein), NF-70 (Neurofilmenet-70) and P tubulin. The results using xenofree media matches well with the observations made by other investigators who worked on different cell types including that from umbilical cord blood stem cells using conventional media [see 17, 23, 27, 28]. These undifferentiated BMMSCs showed a strong neuronal predisposition as revealed by gene expression studies which was also reported earlier [13].

Further, these cells were checked for its functional efficacy in animal models. The 6-OHDA lesioned PD rat model was created to assess the functional efficacy of the differentiated cells. The animals started showing significant behavioral improvement post transplantation, as evaluated by apomorphine induced rotations. One set of animals after completing three months were sacrificed. Their brain sections were analyzed for human cells expressing TH and human nuclei. The results also indicate that the BMMSCs cultured in CBS after injection into a damaged area of the PD rat brain had engrafted and differentiated into functional dopaminergic neurons capable of secreting dopamine and alleviating behavioral deficits. In this respect, MSCS free of animal components to be safe for regenerative medicine, Briefing again: We have injected BMMSC cultured in CBS for neuronal progenitors and injected in to animals to check whether these actually transdifferentiated into neuronal cells. The data which is not shown as mentioned earlier in paragraph [0012] is the behavioral effects which cannot be measured but only recorded as a video clip. To ensure that these BMMSC have the capacity to transdifferentiate into neuronal precursors, the lab experiments were done to provide proof for the neural precursors.

Applicants are not claiming ownership of the source of MSC; however, one of the primary purposes and aim of the present invention is to expand the CD45− and CD73 and 105 positive cells (MSC) from bone marrow and cord blood in a media containing cord blood serum.

CD34− cells are non-hematopoietic cells. The present Application does not have any CD34− marker or cells. If the cells are negative, they are not hematopoietic or designated as non-hematopoietic.

In summary, the present invention demonstrates that BMMSCs are a good potential source for treatment of PD. Since these cells have been grown in CBS, they address the issues raised by translational researchers and clinicians alike. Thus, its infusion into humans for its clinical applications can be well considered.

Thus, the present invention recommends a better and a feasible source of serum, which not only allows expansion of BMMSCs but also maintains and retains the neuronal differentiation potential which will go a long way in the field of regenerative medicine and cell therapy applications with MSCs. These cells can treat such neurodegenerative conditions, where the usual concerns of ethics, infectious disease transmissibility, and immunological reactions etc. will be adequately addressed.

Thus the main features of the invention are:
1. the present invention has successfully achieved derivation of clinical grade MSC from bone marrow under cGMP and strict regulatory conditions;
2. the present invention provides proliferation and neuronal differentiation of the MSCs in a Xenofree cell culture system for clinical applications;
3. the present invention provides a simple two (2) step protocol for neuronal differentiation;
4. the present invention provides MSCs differentiated to neurons, which are characterized at the cellular, molecular and functional level;
5. the present invention has analyzed the in vivo functional efficacy in animal models with human diseases created in a GLP accredited facility;
6. the present invention has accessed the survivability and significant functional improvement in PD animal models as early as two (2) weeks post transplantation, the transplanted cells showed the expression of Dopamine specific TH marker;
7. the results of the present invention re-emphasizes the immense potential of the BMMSC derived in a regulated environment as a replacement therapy for neurodegenerative diseases in general, using PD as representative disease condition; and,
8. the present invention demonstrates that autologous derived BMMSCs are the safest and can be the accepted mode of choice for various cell therapy applications to begin with.

To these ends, the present invention primarily consists in the provision of a method of growing mesenchymal stem cells from bone marrow which comprises the steps of:
a. isolating of a mononuclear cell fraction of bone marrow to provide a mononuclear cell suspension of about 10⁶-10⁷ cells/ml;
b. plating the mononuclear cell suspension of about 10⁶-10⁷ cells/ml into tissue culture flasks comprising a culture medium along with 1-50% cord blood serum for 24-72 hours to produce adhered cell cultures;
c. incubation of the adhered cell cultures of step b. at 37° C. in 5% CO₂ air incubator for at least 7 days to produce cultural cells; then
d. counting and analyzing the cultured cells for expression of markers selected from CD markers; and
e. analyzing of the differentiation potential into neural cells in vitro and in vivo.

The mesenchymal stem cells obtained from bone marrow are positive for CD73, CD105, CD44, CD29 and SSEA4 markers.

The mesenchymal stem cells obtained from bone marrow are negative for CD45, CD31, vWF and CD14 markers.

The mesenchymal stem cells obtained are expressed as positive for MHC class II and are expressed as negative for MHC class I.

The cells are about 90% pure in terms of MSC antigen expression and viability.

The invention further provides for differentiating the mesenchymal stem cells of paragraph [0053] into neural cells and comprises steps of:
a. culturing the Bone Marrow derived MSCs of paragraph [0053], in neuronal preinduction medium for a week;
b. differentiation of the above induced cells into neural cells with antioxidant and protein kinase activator in the same preinduction media for 4-5 hours;
c. characterization of the cells for the expression of neuron specific markers by immunoflourescence and RT-PCR; and
d. in vitro functional assay for secretion of Dopamine.

The preinduction medium referred to in paragraph [0058] is DMEM: F12 (1:1) medium and, comprises 10% CBS, 2% B27, supplemented with growth factors.

The preferred growth factors are 2 ng/ml basic fibroblast growth factor, 100 ng/ml nerve growth factor, and 50 ng/ml of Noggin.

The preferred antioxidant is DMSO.

The preferred protein kinase activator is BHA.

The neural cells referred to in paragraph [0058] are characterized by immunoflourescence and were analyzed for neuronal specific markers selected from NeuN, NF-70, TH, BP, Nestin and GFAP.

The neural cells referred to in paragraph [0058], are characterized by RT PCR and were analyzed for the expression of genes selected from Nestin, NF-H, Beta-tubulin.

The neural cells referred to in paragraph [0058] were analyzed for functional assay for secretion of about 1.93 ng/ml Dopamine by RP-HPLC.

The invention is also further concerned with the use of the mesenchymal stem cells for the treatment of neural disorders and provides for a method of growing mesenchymal stem cells of bone marrow comprising the steps of:
a. isolation of mononuclear cell fraction of bone marrow,
b. plating a mononuclear cell suspension of about 10⁶-10⁷ cells/ml into tissue culture flasks comprising a culture medium along with 1-50% cord blood serum for 24-72 hours to produce adhered cell cultures,
c. incubation of the adhered cell cultures of step b at 37% C in 5% CO₂ air incubator for at least 7 days,
d. counting and analyzing the cultured cells for expression of markers selected from CD markers, and
e. analysis of neural cells derived from bone marrow MSC in vitro and in vivo, and including the step of utilizing the aforesaid mesenchymal stem cells for the treatment of neural disorders.

The method for differentiating the mesenchymal stem cells produced by the aforesaid previous method into neural cells which comprises the steps of:
a. culturing the Bone Marrow derived MSCs of paragraph [0035A] to be changed in a neuronal preinduction medium for a week,
b. differentiation of the above induced cells into neural cells with antioxidant and protein kinase activator in the same preinduction media for 4-5 hours,
c. characterization of the cells for the expression of neuron specific markers by immunoflourescence and RT-PCR, and
d. in-vitro functional assay for secretion of Dopamine, and utilizing the aforesaid neural cells for the treatment of neuronal disorders.

The invention also includes the utilization of βFGF in the range of 1-50 mg/ml, for treatment of neuronal disorders when prepared by the method of culturing human mesenchymal stem cells (hMSC) comprising the steps of culturing said stem cells in a culture medium comprising:
a. Cord Blood Serum in a range of 1-50%;
b. a mixture of: Dulbecco's modified Eagle's medium (DMEM) and Ham's F-12 medium in the range of 1:1; and
c. β-FGF in the range of 1-50 ng/ml, for the aforesaid utilization in the treatment of neuronal disorders.

The invention is also concerned with the use of the method of culturing mesenchymal stem cells (MSC) for therapeutic purposes in regenerative medicine and for cardiac disorders, bone, cartilage and neural disorders, from mononuclear cell fractions of umbilical cord blood or bone marrow wherein the method comprises the steps of:
a) plating a mononuclear cell suspension of about 10⁶-10⁷ cells/ml into tissue culture flasks comprising a culture medium along with 1-50% cord blood serum for 24-72 hours to produce adhered cell cultures;
b) incubating the adhered cell cultures of step a) at 37° C. in 5% CO₂ air incubator for at least 7 days; and
c) counting and analyzing the cultured cells for expression of markers selected from CD73+, CD45− and CD105+ markers.

The invention is also concerned with a method of culturing human mesenchymal stem cells (hMSC) for therapeutic purposes in regeneration medicine and for cardiac disorders, bone, cartilage and neural disorders in the presence of 1-50% cord blood serum as a replacement for fetal bovine serum in a culture medium comprising:
i) obtaining_Cord Blood Serum in a range of 1-50% of cord blood, and
ii) combining i) with a mixture of: Dulbecco's modified Eagle's medium (DMEM) and Ham's F-12 medium in the ratio of 1:1 supplemented with 2 mM glutamine, 3 mM sodium bicarbonate and β-FGF.

The aforesaid culture medium comprises a mixture of: Dulbecco's modified Eagle's medium (DMEM) and Ham's F-12 medium in the ratio of 1:1 supplemented with 2 mM glutamine, 3 mM sodium bicarbonate and β-FGF.

The mesenchymal stem cells are obtained from human bone marrow from human cord blood and from swine bone marrow.

The culture medium expands the mesenchymal stem cells.

The invention is also concerned with the use of the method of culturing mesenchymal stem cells as for therapeutic purposes in regenerative medicine and for cardiac disorders, bone, cartilage and neural disorders.

The invention further includes a method of culturing human mesenchymal stem cells (hMSC) for therapeutic purposes in the presence of 1-50% cord blood serum as a replacement for fetal bovine serum in a culture medium comprising Cord Blood Serum in a range of 1-50% of cord blood, and a mixture of: Dulbecco's modified Eagle's medium (DMEM) and Ham's F-12 medium in the ratio of 1:1 supplemented with 2 mM glutamine, 3 mM sodium bicarbonate and β-FGF.

The method of culturing animal mesenchymal stem cells in a culture medium comprises using Cord Blood serum in a range of 1-50%; and a mixture of: Dulbecco's modified Eagle's medium (DMEM) and Ham's F-12 medium in the ratio of 1:1 supplemented with 2 mM glutamine, 3 mM sodium bicarbonate and β-FGF.

The use of the method of culturing mesenchymal stem cells as for therapeutic purposes in regenerative medicine and for cardiac disorders, bone, cartilage and neural disorders and provides for the use of sera separated from umbilical cord for growing the mesenchymal stem calls, in accordance with the following method, comprises plating a mononuclear cell suspension of about 10⁶-10⁷ cells/ml into tissue culture flasks comprising a culture medium along with 1-50% cord blood serum for 24-72 hours to produce adhered cell cultures, incubation of the adhered cell cultures of step a) at 37° C. in 5% CO₂ air incubator for at least 7 days, and counting and analyzing the cultured cells for expression of markers selected from CD73+, CD45− and CD105+ markers.

The method for using the mesenchymal stem cells for therapeutic purposes in regenerative medicine and for cardiac disorders, bone, cartilage and neural disorders and provides for the use of sera separated from umbilical cord for growing the mesenchymal stem cells.

The Mesenchymal stem cells are for use in regenerative medicine in a neural disorder. The neural disorder is PD.

The CD markers are selected from CD73, CD105, CD44, CD29, SSEA4, CD45, CD31, vWF and CD14.

The mesenchymal stem cells obtained from bone marrow are positive for CD73, CD105, CD44, CD29, SSEA4 markers, and the mesenchymal stem cells obtained from bone marrow are negative for CD45, CD31, vWF and CD14 markers.

The mesenchymal stem cells obtained are further expressed positive for MHC class II and are negative for MHC class I.

The cells are about 90% pure in terms of MSC antigen expression and viability.

The mesenchymal stem cells are for use in regenerative medicine in a neural disorder. The neural disorder is PD.

The invention is also concerned with a method of culturing mesenchymal stem cells (hMSC) and invitro differentiation into neural cells, as noted above and exemplified herein substantially in the examples and figures.

### BRIEF DESCRIPTION OF DRAWINGS

The file of this Patent contains at least one drawing executed in color. Copies of this Patent with color drawings will be provided by the Patent and Trademark Office upon request and payment of the necessary fee.

The following figures, which are in the form of photographs are part of the present specification and are incorporated to further demonstrate certain aspects of the present invention. The invention may be better understood by reference to these figures in combination with the detailed description presented herein.

FIG. 1 Illustrates the growth kinetics and the viability of the BMMSC in xenofree media, and FIG. 1 consists of the following:

FIG. 1a shows the growth kinetics of the BMMSCs expanded in a xenofree media; BMMSC showed an 8-10 fold increase in the cell count after expansion in presence of CBS for a week, and the cells can be expanded for 5 passages; and FIG. 1b is a plot showing the viability of the expanded BMMSCs; and, the viability of the expanded cells was checked at every passage and these cells were more than 90% viable as checked by 7-Aminoactinomycin D (7AAD) on Flow cytometry.

FIG. 2 Illustrates the Immunophenotyping of the BMMSCs expanded in xenofree media by flowcytometry. More specifically, the BMMSCs expanded in CBS media were checked for the cell surface antigen expression for various mesenchymal and haematopoetic markers. The expanded BMMSCs were negative for haematopoeitc marker and strongly expressed mesenchymal markers. The phenotype expressed by the expanded cells was CD73+/CD105+/CD29+/CD44+/SSEA4+/HLA ABC+/HLA DR−/CD45−/CD14−/CD31−/vWF−

FIG. 3 illustrates the Neuronal differentiation of BMMSCs in xenofree media and consists of the following:

FIG. 3a shows the BMMSCs expanded in xenofree medium grow as monolayer, and these undifferentiated cells show a uniform fibroblast like morphology, which are spindle shaped;

FIG. 3b shows the expanded BMMSCs upon exposure to a neuronal induction medium in a xenofree media and illustrate the expanded BMMSCs shows the change in morphology, which begins within four hours of induction;

FIG. 3c shows the condition after four hours when the cytoplasm of the BMMSCs retracts and the cells start acquiring a neuronal morphology with processes; and,

FIG. 3d shows the condition within five hours of neuronal induction the BMMSCs start exhibiting neuronal morphology with processes.

FIG. 4 illustrates the characterization of the neuronal differentiation of BMMSCs by immunoflourscence, and it should be noted that the Undifferentiated BMMSCs were checked for neuron specific markers such as Nestin and β tubulin and the differentiated BMMSCs were checked for other neuronal markers such as Neurofilment-70 (NF-70), Neuron specific nuclear protein (NeuN), Tyrosine Hydrorxylase (TH) and Glial Fibrillary Acidic Protein (GFAP), and FIG. 4 consists of the following:

FIG. 4a shows an expression of Nestin by the undifferentiated BMMSCs;

FIG. 4b shows an expression of β tubulin by the undifferentiated BMMSCs;

FIG. 4c shows an expression of NF-70 by the differentiated BMMSCs;

FIG. 4d shows an expression of NeuN by the differentiated BMMSCs;

FIG. 4e shows an expression of TH by the differentiated BMMSCs; and

FIG. 4f shows an expression of GFAP by the differentiated BMMSCs.

It should also be noted that undifferentiated BMMSCs showed positive staining for Nestin and β tubulin and differentiated cells expressed neuronal specific markers such NF-70, NeuN, TH and GFAP.

FIG. 5 illustrates the Gene expression studies for differentiated BMMSCs to neuronal lineage by Reverse Transcriptase PCR (RT-PCR); the undifferentiated (UD) and the differentiated cells (D) were checked for neuron specific genes with NTERA 2D as positive control; the RT-PCR studies showed that the undifferentiated cells expressed stem cell marker such as Nanog and neuronal genes such as Nestin and β tubulin; and the differentiated cells expressed neuronal and dopaminergic specific genes specific genes such as TH, Nurrl and NFM.

FIG. 6 illustrates the Measurement of dopamine released into the culture supernatant; for this figure, samples were separated on reverse phase nucleosil C18 column and detected with an electrochemical detector, and dopamine levels were calculated using external dopamine standards injected immediately before and after each experiment Representative HPLC chromatogram of dopamine released by differentiated BMMSCs along with standard dopamine.

FIG. 7 Illustrates the Behavioral analysis of the PD animals injected with BMMSCs expanded in xenofree media; the PD animals were subjected to apomorphine; the apomorphine-induced rotations were counted per hour in the transplanted and non transplanted animals; the graph shows the comparison of the rotations in the transplanted and non-transplanted animals for 2, 4, 6, 8, 10, 12 weeks; the transplanted animals showed a significant reduction in the rotations after 4 weeks of BMMSC transplantation; and the transplanted animals continued to show the significant improvement as the weeks progressed.

FIG. 8 Illustrates the Immunohistochemistry of brain sections of PD induced rats after, and consists of the following:

FIG. 8a shows a transverse section of rat brain illustrating the DiI labeled cells in the injection tract 3 months post transplantation; and,

FIG. 8b shows a transverse section of rat brain illustrating positive staining for both TH (FITC) and human nuclei (Alexa 568) confirming the human origin of the transplanted cells.

### DETAILED DESCRIPTION OF THE INVENTION

The term “umbilical cord blood” or “cord blood” is used throughout the specification to refer to blood obtained from a neonate or fetus, most preferably a neonate and preferably refers to blood, which is obtained, from the umbilical cord or placenta of newborns. The use of cord or placental blood as a source of mononuclear cells is advantageous because it can be obtained relatively easily and without trauma to the donor. Cord blood cells can be used for autologous or allogenic transplantation when and if needed. Cord blood is preferably obtained by direct drainage from the umbilical vein.

The term “cell medium” or “cell media” is used to describe a cellular growth medium in which mononuclear cells and/or neural cells are grown. Cellular media are well known in the art and comprise at least a minimum of essential medium plus optional agents such as growth factors, glucose, non-essential amino acids, insulin, transferrin and other agents well known in the art.

The term “non adherent cells” is used to describe cells remaining in suspension in the tissue culture flask at the end of the culture period. The term “adherent cells” is used to describe cells that are attached to the tissue culture plastic, and are detached from the flask by addition of enzyme free cell dissociation buffer from Gibco-BRL or by addition of trypsin-EDTA.

The term “mononuclear cells” is used to describe cells containing a single nucleus isolated from bone marrow (BM) or Umbilical Cord Blood (UCB) using a density gradient of FICOLL™ or PERCOLL™ Mononuclear cells are obtained from bone marrow or umbilical cord blood and are used as a source of Mesenchymal Stem Cells.

As used herein the term “confluent” indicates that the cells have formed coherent cellular monolayer on the surface so that virtually all-available surface is used leading to inhibition of cell proliferation.

In the present invention growth factors are selected from the group consisting of Epidermal growth factor (EGF), Nerve growth factor (NGF), Fibroblast Growth Factor (FGF), Transforming growth factor-β (TGF-β) either singly or in a combination thereof.

In the present invention growth factors are selected from the group consisting of Epidermal growth factor (EGF), Nerve growth factor (NGF), Fibroblast Growth Factor (FGF), Transforming growth factor-β (TGF-β) either singly or in a combination thereof.

In the present invention, culture media is selected from the group consisting of Dulbecco's modified Eagle's medium, (DMEM), Hams F-12 medium, Iscoves modified Dulbeccos medium (IMDM), Roswell Park Memorial Institute medium (RPMI).

The term “Bone marrow derived mesenchymal stem cells” as used herein refers to the mesenchymal stem cells derived from the mononuclear fraction of the bone marrow and is characterized by the expression of CD markers CD73+/CD105+/CD44+/CD29+/SSEA4+/CD45−/CD31−/vWF−/CD14−. These MSC were negative for MHC class I but expressed MHC class II.

The marrow or isolated MSC can be autologous, allogeneic or from xenogeneic sources, and can be embryonic or from post-natal sources. Bone marrow cells may be obtained from iliac crest, femora, tibiae, spine, rib or other medullary spaces. Other sources of MSC include Fat, Periosteum, Skin, and Skeletal muscle, Liver, Placenta, Blood and Umbilical Cord.

The parent application Ser. No. 10/853,077, which is incorporated herein by reference, showed that mesenchymal stem cells could be cultured and expanded in a medium containing CBS. The present invention has shown the neural differentiation of BMMSC expanded in CBS and its in vivo functionality for PD and Spinal cord injury in animal models.

A complete explanation of how these markers are helpful in connection with Parkinson's Disease, as well as any others that are considered pertinent, will be helpful. Table 3 related to the explanation of these markers is provided.

The following steps are involved in the process for derivation of MSCs:
1. isolation of mononuclear cell fraction from the bone marrow by density gradient separation;
2. plating of the cells in culture media for expansion;
3. expansion of the adherent cell cultures till they are confluent;
4. harvesting the cells;
5. characterization of the cells; and,
6. analysis of the differentiation potential into neural cells in vitro and invivo.

1. Cell Isolation

The bone marrow sample is diluted appropriately with PBS. The diluted sample is subjected to density gradient separation using Percoll to obtain mononuclear cells. The mononuclear fraction is plated in cell culture cassettes in MSCGM for expansion of MSC.

2. Expansion

The mononuclear cells recovered are washed with PBS and resuspended in the medium in cell culture cassettes. After forty-eight hours the nonadherent cells are removed and plated into another cell culture cassette. The cells adhere and form colonies, which grow and become a confluent layer. Medium is changed every alternate day until the cells become confluent.

3. Harvesting

Once confluent the cells are harvested using a dissociation buffer to obtain single cells. The washes during the harvest are tested for sterility, bioburden and endotoxin. An aliquot of cells are tested for viability and biomarker expression. The expanded and harvested cells will be placed in transport media in 1.8 ml cryovial and frozen in a control rate freezer to −90° C. and then stored in liquid nitrogen tank.

4. Expansion and Characterization of Bone Marrow Derived MSC Cultured in Medium Containing CBS.

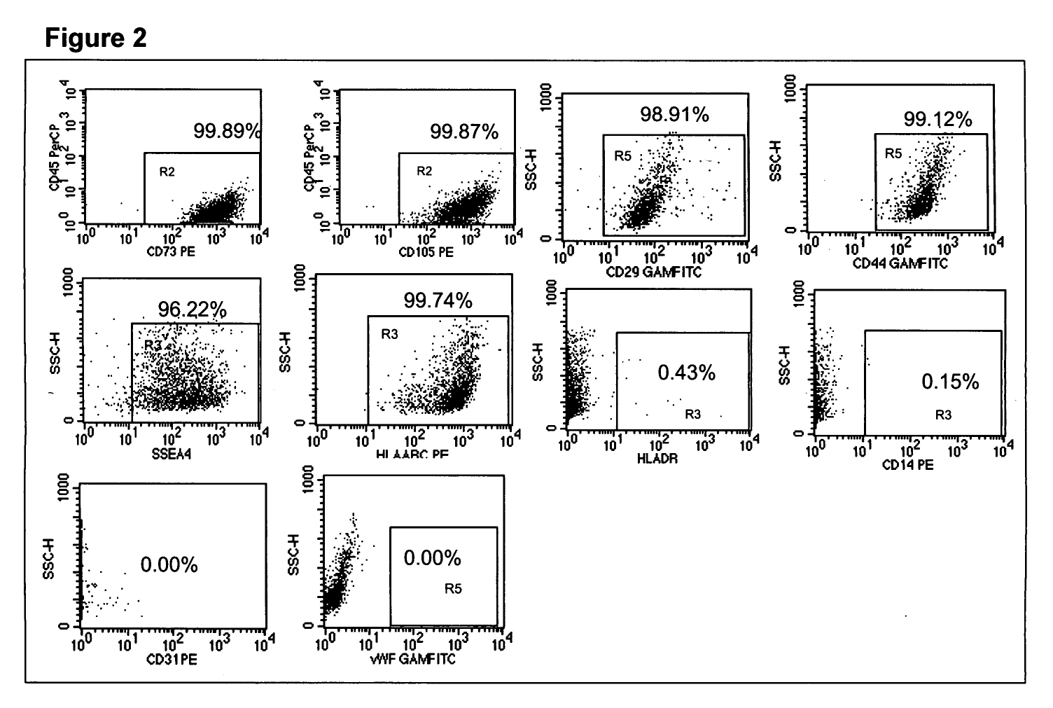

MSCs obtained from human bone marrow were successfully cultured and expanded in medium containing CBS under cGMP conditions. At the passage one, cultures consisted of heterogeneous cells. At passage four, MSC grew as a monolayer of adherent fibroblast like cells and showed a twenty-fold increase in expansion. Furthermore, the results from flow cytometry indicated that undifferentiated MSCs exhibited the positive labeling to mesenchymal markers with CD29, CD105, CD44, CD73 but negative staining to hematopoeitc and endothelial markers (FIG. 2). These cells showed 90% purity in terms of MSC antigen expression and viability and expressed a phenotype of CD73+/CD105+/CD44+/CD29+/SSEA4+/CD45−/CD31−/vWF−/CD14−. These MSC were negative for MHC class I but expressed MHC class II.

5. Neuronal Differentiation and Characterization

Neuronal differentiation was initiated culturing the undifferentiated MSC neuronal pre induction medium containing neurotropic factors and CBS. After one week of culture in neuronal induction medium, the cytoplasm of the cells retracted towards the nucleus. Upon exposure to DMSO/BHA, most cells presented the neuronal morphology including a small cell body and long extensions. To confirm that MSCs differentiated along neuronal lineages we examined the expression of neuronal markers in the cells by immunoflourescence and RT PCR. Immunoflorescence analysis demonstrated that differentiated cells expressed neuronal specific surface markers such as NeuN, NF-70, TH, BT, Nestin and GFAP. Undifferentiated MSC were found Nestin, which was further confirmed by gene expression studies indicating a neural predisposition. RT PCR revealed the expression of characteristic neuronal markers Nestin, NF—H, β tubulin, NF-M, and dopaminergic markers TH and Nurrl. Except for the neuronal markers we also targeted GFAP, a glial marker, GFAP. The number of GFAP positive cells was low. Undifferentiated MSC also expressed Oct-4, the pluripotent stem cell marker. We further evaluated the functional properties of differentiated MSC by RP-HPLC. We found that detectable level of dopamine (1.93 ug/ml) was secreted by the differentiated neurons in to the culture supemetent as compared to undifferentiated MSC (Table 2).

6. In Vivo Differentiation and Function of MSCS Post Transplantation

We next examined in vivo survival, differentiation and function of the undifferentiated MSCs expanded under xeno free and cGMP conditions after transplantation into the substantia nigra of Parkinsonian rats. The PD rat model was created by injecting 6-hydroxydopamine (6-OHDA) into the substantia nigra. The motor abnormality in the PD rat was evaluated by examining rotational behavior in response to apomorphine injection. We selected rats that exhibited stable deterioration of motor functions and showed ≧10 ipsilateral circling per min for cell transplantation. About 0.2 million of MSCs/rat were transplanted into the substantia nigra of rat model of PD six weeks after lesioning. We observed no improvement of motor function during first two weeks post-transplantation, but after a four week period onwards or subsequent, rats showed a significant motor improvement and reduced apomorphine-induced rotations. After a 12-weeks, post-transplanted animals showed a significant reduction in apomorphine induced rotations. The histology of the grafted area showed that transplanted cells survived in the substantia nigra and differentiated into dopaminergic neurons. In order to confirm that transplanted cells were of human origin, double labeling with human nuclei antibody and TH were done. At 12 weeks post transplantation, cells that were immunoreactive with anti human nuclei were found only along the needle tract and within the substantia nigra. There was no indication of cell migration to neighboring brain regions.

Previous studies from our laboratory have shown that culturing of human bone marrow derived Mesenchymal stem cells (MSC) in the presence of human umbilical cord blood serum (CBS) instead of FBS promotes more effective proliferation and also retain their differentiation capacity. In the present study, human bone marrow derived MSC showed a twenty-fold increase in expansion in the presence of CBS over a period of three weeks. These cells showed 90% purity in terms of MSC antigen expression and viability and expressed a phenotype of CD73+/CD105+/CD44+/CD29+/SSEA4+/CD45−/CD31−/vWF−/CD14−. These MSC were negative for MHC class I but expressed MHC class II. They were induced to differentiate into dopaminergic neurons in vitro by culturing in Neuronal induction media supplemented with antioxidant DMSO, protein kinase activator Butylated hydroxy anisole, and growth factors NGF, Noggin that promotes neuronal differentiation. Upon neuronal induction, they expressed proteins specific to neurons as evidenced by immunoreactivity to NeuN, Nestin, Neurofilament-70, β tubulin, GFAP and dopaminergic specific marker TH. These cells expressed neuronal transcripts Nestin, NFH, β tubulin Nurrl, TH, GFAP. Progression towards dopaminergic neuronal fate was also confirmed by the measurement of dopamine released by the cells into the culture supernatant by RP-HPLC. In vivo functionality was assessed by grafting these cells into the substantia nigra of rats previously made Parkinsonian by unilateral lesioning with neurotoxin 6-hydroxy dopamine. There was a significant behavioral improvement in animal models 3 months post transplantation and the injected cells also survived as revealed by immunohistochemical studies of the grafted area with TH and human nuclei.

A complete explanation of how these markers are helpful in connection with Parkinson's Disease, as well as any others that are considered pertinent, will be helpful. A table related to the explanation of these markers is provided and designated Table 3.

Our results suggest that mesenchymal stem cells derived from the human bone marrow can be transdifferentiated efficiently into functional dopaminergic neurons both in vitro and in vivo, and thus offer a viable option for cell-replacement therapy of incurable neurodegenerative diseases including PD.

The present results indicated that bone marrow derived MSCs expanded under xeno free conditions demonstrated the invitro and in vivo neuronal potential and significantly reduced the apomorphine-evoked rotations in parkinsonian rats. BMMSCs, showed a ten (10) fold expansion using xeno free medium, displaying typical MSC phenotype. On differentiation, they exhibited neuronal morphology, expressing cellular and molecular markers adapting a dopaminergic fate. Detectable levels of dopamine (1.93 ng/ml) were observed in the culture supernatants of differentiated cells. There was a significant behavioral improvement in PD models three (3) months post transplantation, substantiated by immunohistochemistry supporting in vivo survival and differentiation.

Further, the present invention has obtained a pure population of BMMSCs propagated to an upscalable clinical quantity with reproducibility, in a clean room environment with our validated process and test methodologies. The present invention demonstrates that BMMSCs can be transdifferentiated efficiently into functional dopaminergic neurons both in vitro and in vivo. This holds immense clinical potential as a replacement therapy for PD and other neurodegenerative diseases as well.

Based on the published data, the present invention has created the rodent disease models for both PD and Spinal cord injury that mimic the clinical symptoms of these diseases to a certain extent. The present invention has assessed these models by behavioral tests before cell transplantation. The efficacy of the bone marrow derived MSC in alleviating the symptoms of the disease was evaluated three (3) months post transplantation by behavioral and histological studies as detailed below.

In this study, the inventors have also demonstrated the dopaminergic differentiation in vitro by BMMSC cultured in CBS by showing TH positive cells by immunoflourescence and dopamine secretion by ECD. In addition to TH the differentiated cells after induction have shown positivity for NeuN, NF-70, BT. They have also shown the ability to form glial cells which is demonstrated by GFAP staining. Undifferentiated BMMSCs cultured in presence have been shown to be positive for nestion suggesting its neural predisposition. Differentiated cells have shown to expression the genes at the RNA, which was confirmed by RT-PCR studies. All these in vitro studies showed a good level of neural differentiation of BMMSC cultured in presence of CBS. Further, these cells were checked for its functional efficacy in animal models. The PD rat model was created to mimic the conditions as humans. After injecting cells in the animals started showing behavioral improvement, which was analyzed by various tests such as water, escape test, front leg placing test apomorphine injection. Appropriate control animals were used to measure the level of improvement behaviorally. The stem cell injected animals were monitored at various intervals and improvement was confirmed accordingly. One set of animals after completing 3 months were sacrificed and their brain sections were analyzed for human cells expressing Th. Also the brain section was checked for differentiated BMMSC co expressing neural markers such as TH and NeuN. Our results also indicate the BMMSCs cultured in CBS after injection into a damaged area of the PD rat brain have engrafted and differentiated into functional dopaminergic neurons which were checked by assaying the serum of the stem cell injected rats.

The following examples are included to demonstrate preferred embodiments of the invention. It should be appreciated by those skilled in the art that the techniques disclosed in the examples which follow represent techniques discovered by the inventor to function well in the practice of the invention, and thus can be considered to constitute preferred modes for its practice. However, those skilled in the art should, in light of the present disclosure, appreciate that many changes can be made in the specific embodiments which are disclosed and still obtain a like or similar result without departing from the spirit and scope of the invention.

### EXAMPLE 1

### Isolation and Expansion of MSC in the Presence of CBS

Human bone marrow from normal volunteers was obtained from the iliac crest after an informed consenting process. The marrow was processed in a clean room environment. Mononuclear cells (MNCs) were isolated as reported earlier [25]. The isolated cells were seeded in 75-cm2 tissue culture flasks (Nunc, USA) in MSC proliferation medium containing DMEM: F12 (1:1) (Invitrogen, Singapore) supplemented with 10% CBS and 1 ng/ml of basic fibroblast growth factor (Sigma, USA), incubated at 37° C. with 5% CO₂. The cells grew as colonies, which then became confluent to form a monolayer. Upon reaching confluency, the cells were harvested using trypsin EDTA (Invitrogen, Singapore) to give a single cell suspension. The harvested cells were analyzed for cell surface markers by flow cytometry (BD Pharmingen, USA).

Results

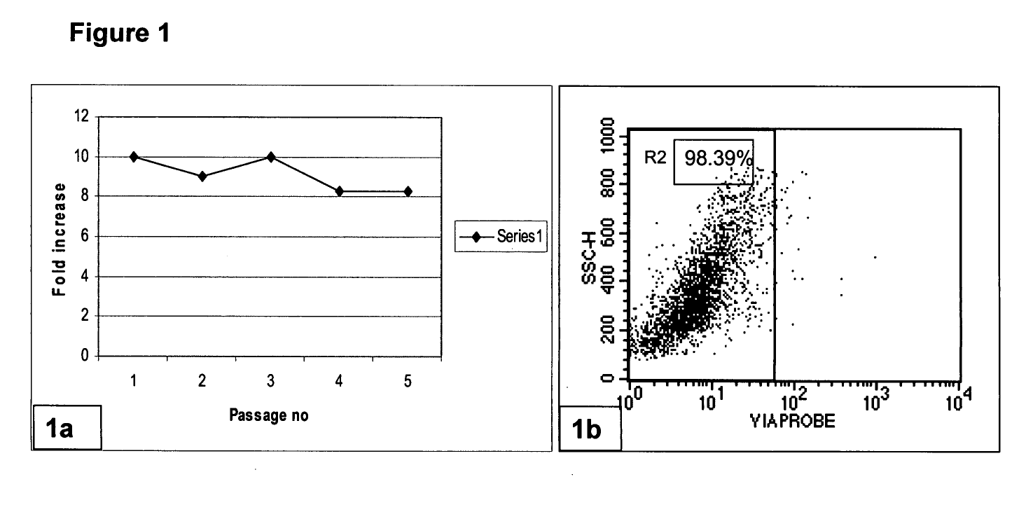

MSCs obtained from human bone marrow were successfully cultured and expanded in medium containing CBS under cGMP conditions. MSCs isolated from bone marrow grew as distinct colonies within 1 week of culture after which, the colonies started expanding and formed a monolayer of adherent fibroblast like cells. MSCs cultured in CBS showed a 8-10 fold increase in expansion. (FIG. 1). Furthermore, the results from flow cytometry indicated that expanded BMMSCs exhibited the positive labeling to mesenchymal markers such as, CD73, CD105, CD44, CD29, but negative staining to hematopoeitc and endothelial markers such as CD45, vWF. (FIG. 2). These cells showed 90% purity in terms of MSC antigen expression and viability and expressed a phenotype of CD73+/CD105+/CD44+/CD29+/SSEA4+/CD45−/CD31−/CD14−/vWF−. (FIG. 2). These MSCs were negative for MHC class II but expressed MHC class I.

### EXAMPLE 2

### Identification of BMMSC Phenotype

Immunophenotyping of the cultured MSCs were done using flow cytometry. The adherent cells were washed with PBS and detached by incubating with 0.05% trypsin EDTA (Invitrogen, Singapore) for 5 minutes at 37° C. The harvested cells were washed using staining buffer containing 4% FBS and 0.1% azide in PBS. After harvesting the adherent cells, a cell count was taken and approximately 50,000 cells were used for cell surface antigen expression studies. Cells were incubated with CD45 PerCP (BD Pharmingen, USA), CD73 PE (BD Pharmingen, USA), CD 105 PE (Caltag), SSEA4 PE (R&D systems, USA), HLADR PE (BD Pharmingen, USA), HLAABC PE (BD Pharmingen, USA), CD14 PE (BD Pharmingen, USA), CD31 PE (BD Pharmingen, USA), CD29 (BD Pharmingen, USA), CD44 (BD Pharmingen, USA), vWF (Santacruz, USA) using standard techniques [24]. Goat anti mouse FITC was used as a secondary antibody to detect the vWF primary antibody. Appropriate isotype controls were used. These cells were acquired on a FACS Calibur Flow Cytometer (BD Pharmingen, USA) equipped with a 488 nm Argon Laser. Approximately 10,000 events were acquired and analyzed using Cell Quest Software. For viability determination, cells were stained with 7-Amino Actinomycin D (7-AAD), (BD Pharmingen, USA) & acquired on the flow cytometer. Dead cells take up the fluorescent stain while live cells exclude this stain. Viability and surface antigen expression were evaluated at every passage.

### EXAMPLE 3

### Neural Differentiation of MSC Cultured in CBS

For inducing the neuronal differentiation, a modified version of Woodbury et al., protocol was followed. Briefly, after 3 days of expansion in MSC proliferation medium, the MSCs were pre induced in DMEM: F12 (1:1) medium (Invitrogen, Singapore) containingl 0% CBS, 2% B27 (Sigma, USA) and supplemented with growth factors 2 ng/ml basic fibroblast growth factor (Sigma, USA), 100 ng/ml nerve growth factor, 50 ng/ml of Noggin (Peprotech, USA). The cells were maintained in neuronal pre induction medium for a week with media changes affected every alternate day. After a week, the cells were induced with 200 μM BHA (Sigma, USA) in the same media for 4-5 hours to adapt the dopaminergic fate. Differentiated cells were characterized for the expression of neuron specific markers by immunoflourescence and RT-PCR. For characterization studies the expanded cells were plated in 8 well chamber slides (BD Falcon, USA) at a density of 3000 cells per well. The expanded BMMSCs were also seeded in 35 mm petridishes at a density of 3000 cells per plate for RT-PCR and in vivo transplantation studies in animal models. Controls included cells, which were cultured in MSC proliferation medium for a week.

Results

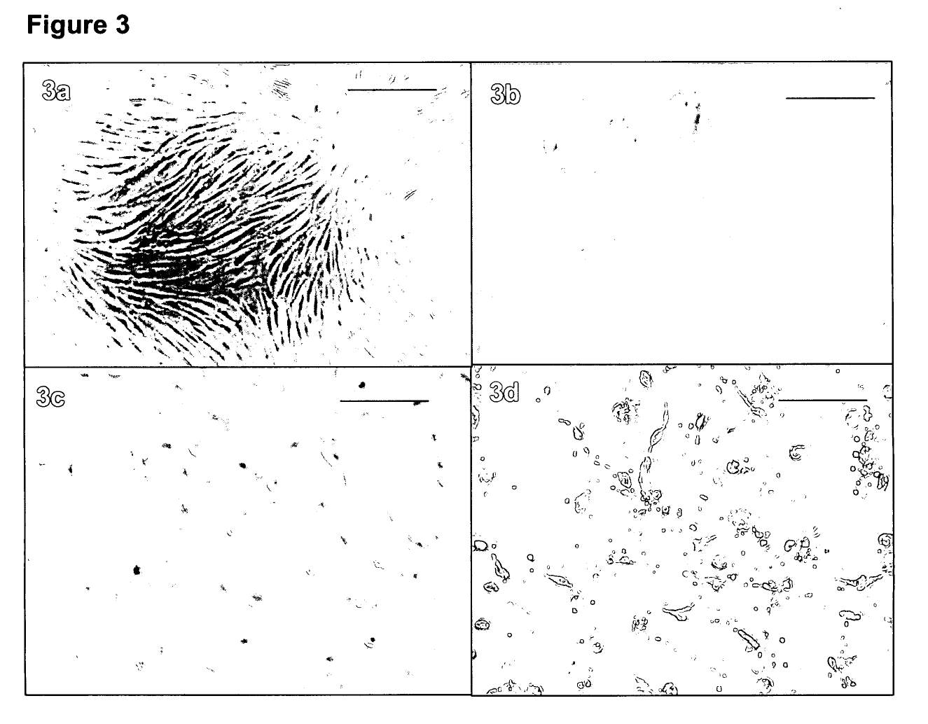

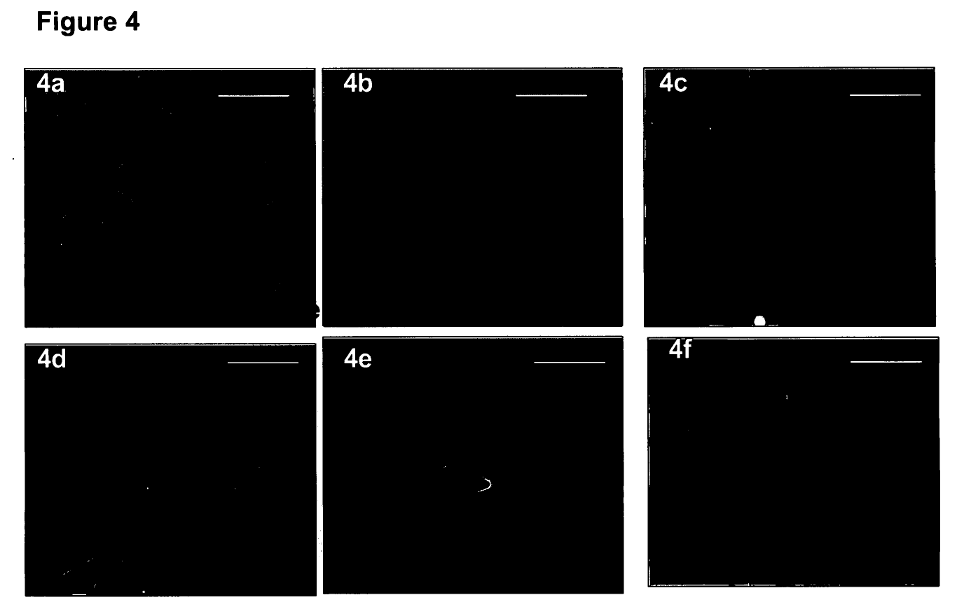

Neuronal differentiation was initiated by culturing the undifferentiated BMMSCs in neuronal pre induction medium containing neurotropic factors and CBS. After one week of culture in neuronal induction medium, the cytoplasm of the cells retracted towards the nucleus. Upon exposure to strong neural inducers such as DMSO/BHA, most cells presented the neuronal morphology including a small cell body and long processes. To confirm that BMMSCs differentiated along neuronal lineages we examined the expression of specific markers in the cells by immunoflourescence and RT PCR. Immunoflourescence analysis demonstrated that differentiated cells expressed neuronal specific surface markers such as NeuN, NF-70, TH, and β tubulin, Nestin and GFAP (FIG. 3). Undifferentiated BMMSCs were found to express Nestin and P tubulin, which was further confirmed by gene expression studies indicating a neuronal predisposition. RT PCR revealed the expression of characteristic neuronal markers Nestin, NFH, β tubulin, NFM, and dopaminergic markers TH, Nurrl (FIG. 4). We also targeted GFAP, a glial marker. The number of GFAP positive cells was low. Undifferentiated BMMSCs also expressed nanog but were negative for OCT4 (FIG. 4). We further evaluated the functional properties of differentiated BMMSCs by RP-HPLC. We found that detectable level of dopamine (1.93 ng/ml) was secreted by the differentiated neurons in to the culture supernatant as compared to undifferentiated BMMSCs (Table 1).

TABLE 1
DOPAMINE CONCENTRATION (NG/ML) IN CULTURE
SUPERNATANT BY RP-HPLC

| | | Number of | | | Concentration |
|---|---|---|---|---|---|
| | | samples | No of days in | Area covered | of dopamine |
| # | Culture conditions | analyzed | culture | by the peak | (ng/ml) |
| 1 | MSC expanded in 10% CBS | 3 | 19 | 969 | 0.03 |
| | and DMEM/F12 | | | | |
| 2 | MSC differentiated in | 3 | 7 | 56446 | 1.94 |
| | neuronal induction media | | | | |
| | along with DMSO/BHA | | | | |

### EXAMPLE 4

### Immunofluorescence Studies

In order to confirm that MSCs differentiated into neuronal lineage, the protein markers expressed by the differentiated cells were identified by immunoflourescence. After 4-5 hours of induction with BHA in neuronal media, the cells grown on 8 well chamber slides were washed with 1×PBS and fixed with 4% paraformaldehyde at 4° C. for 30 minutes. Then the cells were rinsed with PBS and stained with neuronal markers. The differentiated cells were checked for expression of the following antibodies: Glial fibrillary acidic protein (GFAP, 1:100), Nestin (1:200), Neuronal specific nuclear protein (NeuN, 1:200), Neurofilament-70 (NF-70, 1:100), β tubulin (1:100), Tyrosine hydroxylase (TH, 1:100). All the primary antibodies were procured from Chemicon, USA. The primary antibodies were prepared in staining buffer consisting of 0.1% Triton X-100 in 1×PBS. The cells were then incubated overnight at 4° C. with primary antibody. After washing three times with 1×PBS, cells were incubated with goat antimouse Alexa 488 (1:500, Molecular probes) as a secondary antibody for 30 minutes at 37° C. and counter stained with DAPI (1 μg/ml, Sigma). Immunopositive areas were looked for by using a fluorescence microscope (Nikon Eclipse E600)

### EXAMPLE 5

### Gene Expression Studies by RT-PCR

The cell pellets of both induced and uninduced cells were used for total RNA extraction. Total RNA was isolated from the 1×10⁶ cells by Trizol method. (Invitrogen, Singapore). 5 μg of RNA was used for cDNA synthesis. The cDNA was synthesized using Superscript reverse-transcriptase II (Invitrogen, Singapore). 1 μl of cDNA was amplified by polymerase chain reaction using ABgene 2×PCR master mix (ABgene, UK) with appropriate primers. The list of primers is as given in Table 2. Cycling parameters are as follows: Initial denaturation at 94° C. for 2 minutes, denaturation at 94° C. for 30 seconds, annealing at 55-65° C. for 30 seconds depending on the primer and elongation for 1 minute and the number of cycles varied between 25 and 40. Final elongation was carried out at 72° C. for 7 minutes.

TABLE 2
PRIMER SEQUENCES USED FOR PCR

| | | | Annealing | |
|---|---|---|---|---|
| Gene | Forward | Reverse | Temp | Size |
| GAPDH | TGAAGGTCGGAGTCAACGGATTTGG | CATGTGGGCCATGAGGTCCACCAC | 60° C. | 890 |
| Nanog | CCTCCTCC TGGATCTG TTATTC A | CAG GTCTTCACCTGTTTG TAG CTG AG | 52° C. | 262 |
| OCT4 | CGRGAAGCTGGAGAAGGAGAAGCTG | CAAGGGCCGCAGCTTACACATGTTC | 58° C. | 247 |
| Nestin | TTTTCCACTCCAGCCATCC | CCAGAACTCAAGCACCAC | 58° C. | 295 |
| GFAP | TCATCGCTCAGGAGGTCCTT | CTGTTGCCAGAGATGGAGGTT | 65° C. | 400 |
| β Tubulin | CTTACTACTGTTAGATCCCAGGAAT | TGAGACGATGTCCTCCATA | 56° C. | 240 |
| TH | TCATCACCTGGTCACCAAGTT | GGTCGCCGTGCCTGTACT | 62° C. | 107 |
| Nurr1 | CGGACAGCAGTCCTCCATTAAGGT | CTGAAATCGGCAGTACTGACAGCG | 68° C. | 790 |
| NF-M | GAG CGC AAA GAC TAC CTG AAG A | CAG CGA TTT CTA ATC CAG AGC C | 63° C. | 430 |

### EXAMPLE 6

### Functional: Dopamine Measurement by HPLC

The functional capacity of both induced and uninduced BMMSCs were evaluated by measuring the dopamine release into the culture supernatant after one week of differentiation forth-eight (48) hours after the last medium change) by Reverse phase —HPLC. Culture supernatants from undifferentiated and 1-week post differentiation were immediately stabilized after collection with 7.5% orthophosphoric acid/metabisulphite (0.22 mg/ml) and stored at 80° C. until analysis. The mobile phase consisted of sodium acetate (0.2M), EDTA (0.2 mM), heptane sulfonic acid (0.55%), dibutylamine (0.01%) and methanol (16%). The pH was adjusted to 3.92 with orthophosphoric acid. Samples (100 μl) were separated on reverse phase nucleosil C18 column and detected with an electrochemical detector. The mobile phase was pumped at a flow rate of 0.5 ml/min. Dopamine levels were calculated using external reference dopamine standards injected immediately before and after each experiment

### EXAMPLE 7

### In Vivo Studies

A. Creation of PD Rat Model (PD)

Rat model of PD was created according to Ravindran and Rao, 2006 [26]. Briefly adult male Sprague Dawley rats weighing about 180-250 g (n=25) were anesthetized with ketamine (50 mg/kg i.p) and Valium (30 mg/kg i.p) and fixed in a stereotaxic frame (Stoelting Co. USA). Ten microlitres of 6-hydroxydopamine (6-OHDA) (6 mg/ml) was injected using motorized microinjector at the flow rate of 1 ul/minute, lowered into the substantia nigra using stereotaxic-guided coordinates [from Bregma 4.5 mm, left 2.2 mm, and ventral 7.8 mm]. 4-6 weeks after 6-OHDA lesioning, animals were examined for rotational symmetry after i.p. Injection of 3 mg/kg of apomorphine hydrochloride (Sigma, USA). Rats showing ≧10 rotations per minute over one hour interval were selected and randomly assigned to treatment or control groups. To prevent subjective bias, a trained examiner unaware of the experimental details performed the evaluation. All animal studies were performed as per Committee for the Purpose of Control and Supervision of Experimental Animals (CPCSEA) guidelines and were approved by our Institutional Animals Ethics Committee.

B. Transplantation of MSC Cultured in Medium Containing CBS

The 6-OHDA lesioned rats, which showed significant ipsilateral turning response, were selected for the study. The rats (n=12), were used for cell transplantation. Each rat was anesthetized with ketamine (50 mg/kg i.p) and valium (30 mg/kg i.p) and fixed in a stereotaxic frame (Stoelting Co. USA) and received an injection of 0.2-0.3 million undifferentiated BMMSCs suspended in the 10 μl of medium using 28-gauge syringe at the rate of 2 ul/min into the substantia nigra. All the cells injected were labeled with cell tracker dye such as DiI (molecular probes) prior to transplantation. Sham-operated control rats (n=6) underwent the same procedure except that they received only saline. Severity of the disease and the extent of recovery were assessed in Sham-injected and cell transplanted animals by physical activity and behavioral response. Rotations were counted for one hour after subcutaneous injection of apomorphine hydrochloride (Sigma, USA). At the end of three (3) months all the animals were evaluated by apomorphine induced rotations and histology studies.

Immunohistochemical Analysis of Pd Brain Injected with MSC Cultures in CBS

12 weeks post transplantation; rats were anesthetized with ketamine (50 mg/kg i.p) and valium (30 mg/kg i.p) and perfused with saline followed with 4% paraformaldehyde. The brains were equilibrated in 20% sucrose in PBS overnight at room temperature. The brains were processed to obtain thin paraffin sections approximately 4-10μ for immunohistological studies. The sections were deparaffinized by xylene and ethanol treatments followed by a subsequent antigen retrival by dipping the slides in citrate buffer. The slides were heated in a microwave for 30 seconds and permeabilized with 0.2% Triton X-100 in PBS. The non-specific binding slides were blocked with 1% BSA in PBS. The sections were then incubated overnight at 4° C. with anti-TH and anti-human nuclei (Chemicon, USA) antibodies along with negative controls. The sections were then washed with PBS and incubated with the appropriate secondary antibody conjugate. To confirm the presence of transplanted TH positive cells, co-localization with anti-human nuclei was done. The sections were embedded in immunoflour mounting medium and observed under a fluorescence microscope (Nikon Eclipse E600) to check for immunopositive cells.

Results

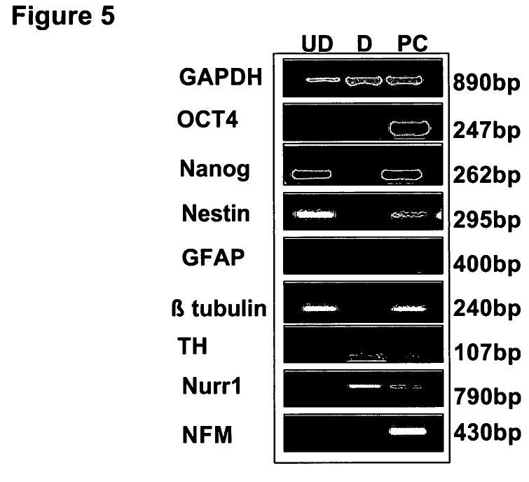

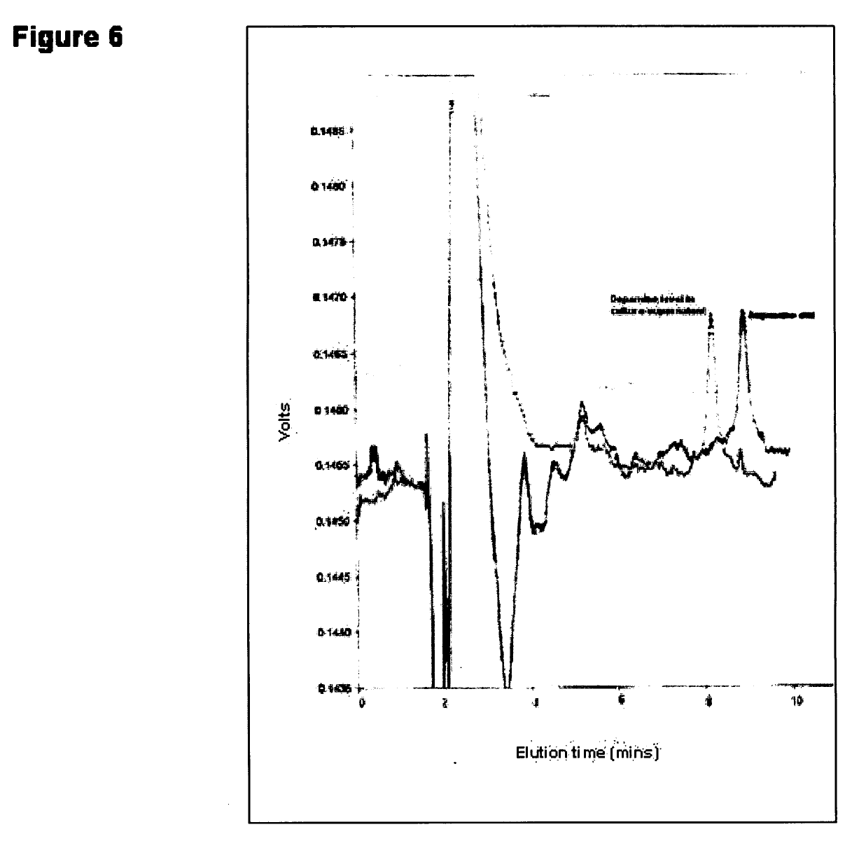

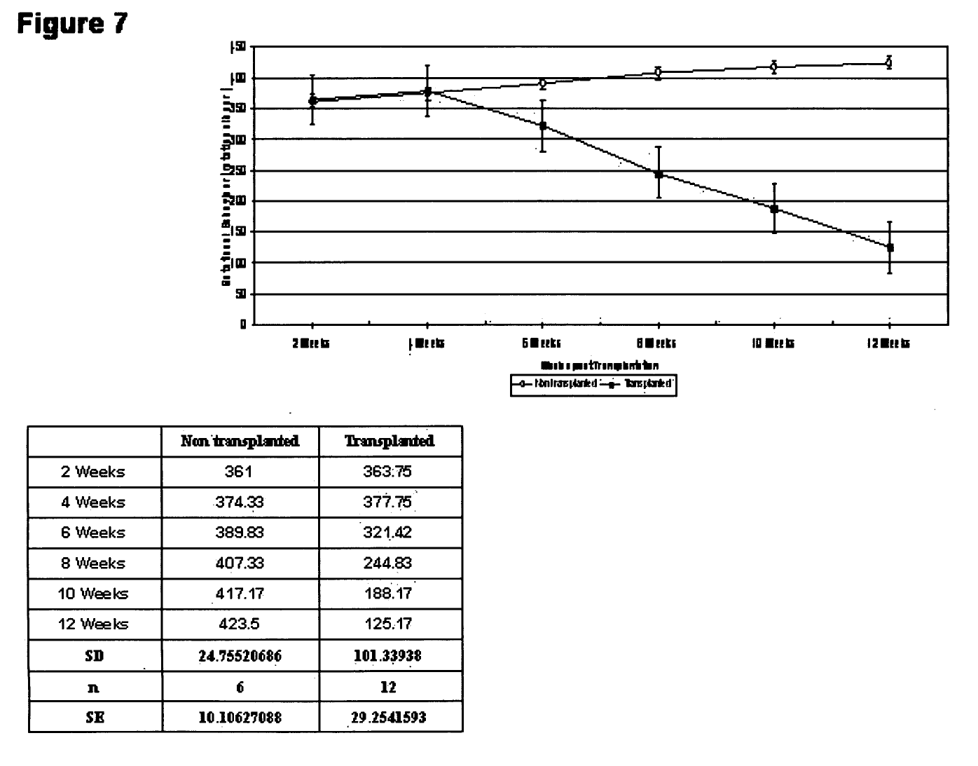

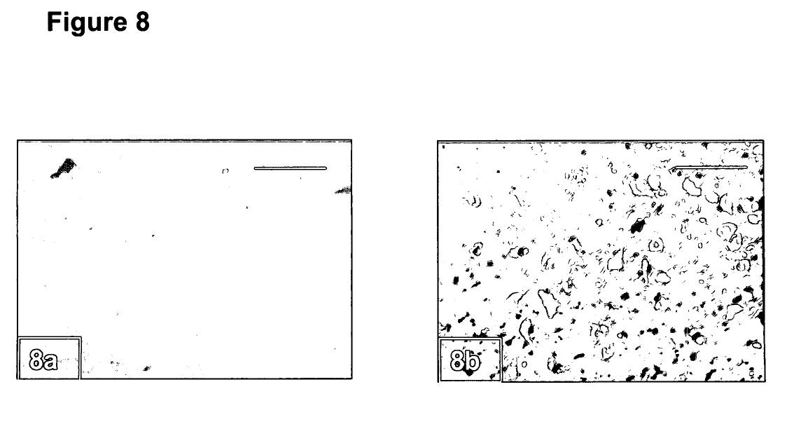

We then examined in vivo survival, differentiation and function of the undifferentiated BMMSCs expanded under xeno free and cGMP conditions after transplantation into the substantia nigra of Parkinsonian rats created in a GLP accredited facility. The PD rat model was created by injecting 6-OHDA into the substantia nigra. The motor abnormality in the PD rat was evaluated by examining rotational behavior in response to apomorphine injection. We selected rats that exhibited stable deterioration of motor functions and showed ≧10 ipsilateral circling per minute for cell transplantation. About 0.2-0.3 million of BMMSCs per rat were transplanted into the substantia nigra of rat model of PD six weeks after lesioning. We observed no improvement of motor function during first two weeks post-transplantation, but after four week onwards, rats showed a significant motor improvement and reduced apomorphine-induced rotations. After 12-weeks, post-transplanted animals showed a significant reduction in apomorphine induced rotations (FIG. 5). The histology of the grafted area showed that transplanted cells survived in the substantia nigra and differentiated into dopaminergic neurons. In order to confirm that the transplanted cells were of human origin, double labeling with human nuclei antibody and TH were done. At 12 weeks post transplantation, cells that were immunoreactive with anti human nuclei were observed along the injection tract and within the substantia nigra.

The following table 3 of markers is provided.

TABLE 3
MARKERS

| | | Positive/ | |
|---|---|---|---|
| Markers | Identification | Negative | Indicates |
| OCT 4 | Embryonic stem cell | −ve | The cells are of |
| | marker | | adult source and not |
| | | | embryonic like. |
| CD34, CD45 | Hematopoietic | −ve | The cells are non- |
| | marker | | hematopoietic. |
| CD73, Cd105, | Mesenchymal stem | +ve | The cells are |
| Cd44, CD29 | cells | | mesenchymal |
| vWF | Endothelial markers | −ve | |
| SSEA4 | Undifferentiated | +ve | The cells are |
| | | | undifferentiated |
| HLA ABC | MHC class I | +ve | Expressed by all the |
| | | | cells (immature as |
| | | | well as mature) |
| HLA DR | MHC class II | −ve | Expressed by |
| | | | matured cells. |
| CD73 | Stem cell marker, | +ve | Stem cell marker |
| | endothelial and | | |
| | epithelial. | | |
| CD105 | Undifferentiated | +ve | This marker is |
| | | | expressed by stem |
| | | | cell precursors of |
| | | | mesenchymal origin |
| CD14 | Differentiated | −ve | This marker is |
| | | | expressed by |
| | | | monocytes, |
| | | | macrophages and |
| | | | granulocytes. |
| CD 29 | Differentiated | +ve | This marker is |
| | | | expressed by T |
| | | | cells, B cells, NK |
| | | | cells, macrophage, |
| | | | monocyte, |
| | | | Endothelial and |
| | | | epithelial. |
| CD31 | Differentiated | −ve | This marker is |
| | | | expressed by T cell, |
| | | | B cell, NK cell, |
| | | | macrophage, |
| | | | monocyte, |
| | | | granulocyte, platelet |
| | | | and endothelial |

For the sake of explanation and use of conventional designation, slant lines [1] are used to separate the markers. “/” slant lines are used to separate markers expressed by the cells which are generally written in this form. The slant lines indicates “and” and not “or” as in usual or conventional American or English. Slant lines are indicative of “commas”. This type of format is preferred by the assignee and the inventors, since it is their normal conventional usage, and such usage is preferred. For example, the cells are CD73+/CD105+ and this indicates that cells are positive for CD 73 and CD105 markers.

The + and − signs are used at appropriate places. The + sign indicates that the cells mark positive for that respective marker and − sign indicates the cells do not/are negative for the marker. Wherever these signs are absent, we have collectively mentioned in that para the cells are positive for [PLEASE COMPLETE] markers and are negative for [PLEASE COMPLETE]. The examiner (cell biologist) can easily decipher this denominations and hence there should be no change in the way it is written.

It should be noted that typically, MNCs comprise hematopoietic and non-hematopoietic cells. This invention uses the non-hematopoietic cells. Initially the cells while isolated from MNC, they are positive for CD markers: CD45, CD34. Later on during expansion, the cells are positive and negative for CD73+, CD 105+, CD44+, CD29+, CD45−, CD34−. And, during differentiation, the cells are positive and negative for CD73+, CD105+, CD44+, CD29+, CD45−, CD34−. They do express the mesenchymal markers in addition to the neuronal markers such as Nestin, Beta tubulin, NCAM, and Neurofilament etc.

Further, it should be noted that cells CD45 is equivalent to CD34, both are hematopoietic cell markers, if they are negative then the cells are non-hematopoietic cells.

All of the compositions and methods disclosed and claimed herein can be made and executed without undue experimentation in light of the present disclosure. While the compositions and methods of this invention have been described in terms of preferred embodiments, it will be apparent to those of skill in the art that variations and modifications may be applied to the compositions and/or methods and in the steps or in the sequence of steps of the methods described herein without departing from the concept, spirit and scope of the invention. More specifically, it will be apparent to those skilled in the art that certain agents that are chemically or physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of growing mesenchymal stem cells from bone marrow comprising steps of:
a. isolation of mononuclear cell fraction of bone marrow,
b. plating a mononuclear cell suspension of about 10⁶-10⁷ cells/ml into tissue culture flasks comprising a culture medium along with 1-50% cord blood serum for 24-72 hours to produce adhered cell cultures,
c. incubation of the adhered cell cultures of step b) at 37° C. in 5% CO₂ air incubator for at least 7 days,
d. counting and analyzing the cultured cells for expression of markers selected from CD markers, and
e. analysis of neural cells derived from bone marrow MSC in vitro and in vivo.

2. The method as claimed in claim 1, wherein the CD markers are selected from the group consisting of CD73, CD105, CD44, CD29, SSEA4, CD45, CD31, vWF and CD14.

3. The method as claimed in claim 2, wherein the mesenchymal stem cells obtained from bone marrow are positive for CD73, CD105, CD44, CD29, SSEA4 markers.

4. The method as claimed in claim 2, wherein the mesenchymal stem cells obtained from bone marrow are negative for CD45, CD31, vWF and CD14 markers.

5. The method as claimed in claim 1, wherein the mesenchymal stem cells obtained further expressed positive for MHC class II and are negative for MHC class 1.

6. The method as claimed in claim 1, wherein the cells are about 90% pure in terms of MSC antigen expression and viability.

7. A method for differentiating the mesenchymal stem cells produced by the method according to the method of claim 1 into neural cells comprising the steps of:
a. culturing the Bone Marrow derived MSCs of claim 1, in a neuronal preinduction medium for a week,
b. differentiation of the above induced cells into neural cells with antioxidant and protein kinase activator in the same preinduction media for 4-5 hours,
c. characterization of the cells for the expression of neuron specific markers by immunoflourescence and RT-PCR, and
d. in-vitro functional assay for secretion of Dopamine.

8. The method according to claim 7, wherein the preinduction medium is DMEM: F12 (1:1) medium, comprising 10% CBS, 2% B27, supplemented with growth factors.

9. The method according to claim 8, wherein the growth factors are 2 ng/ml basic fibroblast growth factor, 100 ng/ml nerve growth factor, and 50 ng/ml of Noggin.

10. The method according to claim 7, wherein the antioxidant is DMSO.

11. The method according to claim 7, wherein the protein kinase activator is BHA.

12. The method according to claim 7, wherein the neural cells characterized by immunofluororesence were analyzed for neuronal specific markers selected from the group of NeuN, NF-70, TH, BP, Nestin and GFAP.

13. The method according to claim 7, wherein the neural cells characterized by RT PCR were analyzed for the expression of genes selected from Nestin, NF-H, Beta-tubulin.

14. The method according to claim 7, wherein the functional assay of the neural cells were analyzed for secretion of about 1.93 ng/ml Dopamine by RP-HPLC.

15. The method according to claim 1, wherein the said Mesenchymal stem cells are for use in regenerative medicine in a neural disorder.

16. A method of growing mesenchymal stem cells from bone marrow comprising steps of:
a. isolation of mononuclear cell fraction of bone marrow,
b. plating a mononuclear cell suspension of about 10⁶-10⁷ cells/ml into tissue culture flasks comprising a culture medium along with 1-50% cord blood serum for 24-72 hours to produce adhered cell cultures,
c. incubation of the adhered cell cultures of step b at 37% C in 5% CO₂ air incubator for at least 7 days,
d. counting and analyzing the cultured cells for expression of markers selected from CD markers, and
e. analysis of neural cells derived from bone marrow MSC in vitro and in vivo, and including the step of utilizing said mesenchymal stem cells for treatment of neural disorders.

17. The method for differentiating the mesenchymal stem cells produced by the method according to the method of claim 16 into neural cells comprising the steps of:
a. culturing the Bone Marrow derived MSCs of claim 1, in a neuronal preinduction medium for a week,
b. differentiation of the above induced cells into neural cells with antioxidant and protein kinase activator in the same preinduction media for 4-5 hours,
c. characterization of the cells for the expression of neuron specific markers by immunoflourescence and RT-PCR, and
d. in vitro functional assay for secretion of Dopamine, and utilizing the neural cells for the treatment of neuronal disorders.

18. A method of culturing human mesenchymal stem cells (hMSC) comprising the steps of culturing said stem cells in a culture medium comprising:
i) Cord Blood Serum in a range of 1-50%;
ii) a mixture of: Dulbecco's modified Eagle's medium (DMEM) and Ham's F-12 medium in the range of 1:1; and
iii) β-FGF in the range of 1-50 ng/ml, for utilization in the treatment of neuronal disorders.

19. The use of the method of culturing mesenchymal stem cells as claimed in claim 1 for therapeutic purposes in regenerative medicine and for cardiac disorders, bone, cartilage and neural disorders.


SEQUENCE LISTING (18 sequences)

SEQ ID NO: 1 - DNA, 25 bp, Homo sapiens
  misc_feature: mesenchymal
tgaaggtcggagtcaacggatttgg

SEQ ID NO: 2 - DNA, 24 bp, Homo sapiena
  misc_feature: mesenchymal
catgtgggccatgaggtccaccac

SEQ ID NO: 3 - DNA, 23 bp, Homo sapiens
  misc_feature: mesenchymal
cctcctcctggatctgttattca

SEQ ID NO: 4 - DNA, 26 bp, Homo sapiens
  misc_feature: mesenchymal
caggtcttcacctgtttgtagctgag

SEQ ID NO: 5 - DNA, 25 bp, Homo sapiens
  misc_feature: mesenchymal
cgrgaagctggagaaggagaagctg

SEQ ID NO: 6 - DNA, 25 bp, Homo sapiens
  misc_feature: mesenchymal
caagggccgcagcttacacatgttc

SEQ ID NO: 7 - DNA, 19 bp, Homo sapiens
  misc_feature: mesenchymal
ttttccactccagccatcc

SEQ ID NO: 8 - DNA, 19 bp, Homo sapiens
  misc_feature: mesenchymal
ccagaaactcaagcaccac

SEQ ID NO: 9 - DNA, 20 bp, Homo sapiens
  misc_feature: mesenchymal
tcatcgctcaggaggtcctt

SEQ ID NO: 10 - DNA, 21 bp, Homo sapiena
  misc_feature: mesenchymal
ctgttgccagagatggaggtt

SEQ ID NO: 11 - DNA, 25 bp, Homo sapiens
cttactactgttagatcccaggaat

SEQ ID NO: 12 - DNA, 19 bp, Homo sapiens
tgagacgatgtcctccata

SEQ ID NO: 13 - DNA, 21 bp, Homo sapiens
  misc_feature: mesenchymal
tcatcacctggtcaccaagtt

SEQ ID NO: 14 - DNA, 18 bp, Homo sapiens
  misc_feature: mesenchymal
ggtcgccgtgcctgtact

SEQ ID NO: 15 - DNA, 24 bp, Homo sapiens
  misc_feature: mesechymal
cggacagcagtcctccattaaggt

SEQ ID NO: 16 - DNA, 24 bp, Homo sapiens
  misc_feature: mesenchymal
ctgaaatcggcagtactgacagcg

SEQ ID NO: 17 - DNA, 22 bp, Homo sapiens
  misc_feature: mesenchymal
gagcgcaaagactacctgaaga

SEQ ID NO: 18 - DNA, 22 bp, Homo sapiens
  misc_feature: mesenchymal
cagcgatttctaatccagagcc